SEQUENCE LISTING (13 sequences)

SEQ ID NO: 1 - protein, 11 aa, Artificial Sequence
  Description of Artificial Sequence Synthetic peptide
KASQDVSTAVA

SEQ ID NO: 2 - protein, 7 aa, Artificial Sequence
  Description of Artificial Sequence Synthetic peptide
LTSYRNT

SEQ ID NO: 3 - protein, 9 aa, Artificial Sequence
  Description of Artificial Sequence Synthetic peptide
QQHYKTPYS

SEQ ID NO: 4 - protein, 5 aa, Artificial Sequence
  Description of Artificial Sequence Synthetic peptide
GYGIY

SEQ ID NO: 5 - protein, 16 aa, Artificial Sequence
  Description of Artificial Sequence Synthetic peptide
MIWGDGRTDYNSALKS

SEQ ID NO: 6 - protein, 8 aa, Artificial Sequence
  Description of Artificial Sequence Synthetic peptide
DYYGSKDY

SEQ ID NO: 7 - protein, 625 aa, Homo sapiens
MIFLYQVVHFILFTSVSGECVTQLLKDTCFEGGDITTVFTPSAKYCQVVCTYHPRCLLFTFTAESPSEDPTRWFTCVLKDSVTETLPRVNRTAAISGYSFKQCSHQISACNKDIYVDLDMKGINYNSSVAKSAQECQERCTDDVHCHFFTYATRQFPSLEHRNICLLKHTQTGTPTRITKLDKVVSGFSLKSCALSNLACIRDIFPNTVFADSNIDSVMAPDAFVCGRICTHHPGCLFFTFFSQEWPKESQRNLCLLKTSESGLPSTRIKKSKALSGFSLQSCRHSIPVFCHSSFYHDTDFLGEELDIVAAKSHEACQKLCTNAVRCQFFTYTPAQASCNEGKGKCYLKLSSNGSPTKILHGRGGISGYTLRLCKMDNECTTKIKPRIVGGTASVRGEWPWQVTLHTTSPTQRHLCGGSIIGNQWILTAAHCFYGVESPKILRVYSGILNQSEIKEDTSFFGVQEIIIHDQYKMAESGYDIALLKLETTVNYTDSQRPICLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 8 - protein, 116 aa, Artificial Sequence
  Description of Artificial Sequence Synthetic polypeptide
QVQLQESGPGLVKPSETLSITCTVSGFSLTGYGIYWVRQPPGKGLEWLGMIWGDGRTDYNSALKSRVTISKDNSKSQVSLKLSSVTAADTARYYCARDYYGSKDYWGQGTTVTVSS

SEQ ID NO: 9 - protein, 107 aa, Artificial Sequence
  Description of Artificial Sequence Synthetic polypeptide
DIQMTQSPSSLSASVGDRVTITCKASQDVSTAVAWYQQKPGKAPKLLIYLTSYRNTGVPDRFSGSGSGTDFTFTISSLQPEDIAVYYCQQHYKTPYSFGGGTKVEIK

SEQ ID NO: 10 - protein, 214 aa, Artificial Sequence
  Description of Artificial Sequence Synthetic polypeptide
DIQMTQSPSSLSASVGDRVTITCKASQDVSTAVAWYQQKPGKAPKLLIYLTSYRNTGVPDRFSGSGSGTDFTFTISSLQPEDIAVYYCQQHYKTPYSFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 443 aa, Artificial Sequence
  Description of Artificial Sequence Synthetic polypeptide
QVQLQESGPGLVKPSETLSITCTVSGFSLTGYGIYWVRQPPGKGLEWLGMIWGDGRTDYNSALKSRVTISKDNSKSQVSLKLSSVTAADTARYYCARDYYGSKDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 705 bp, Artificial Sequence
  Description of Artificial Sequence Synthetic polynucleotide
atgagggtccccgctcagctcctggggctcctgctgctctggctcccaggcgcgcgatgtgacatccagatgacccagtctccatcctccctgtctgcatctgtaggagacagagtcaccatcacttgcaaggccagtcaggatgtgagtactgctgttgcctggtatcagcagaaaccagggaaagcccctaagctcctgatctacttgacatcctaccggaacactggggtcccagataggttcagtggaagtggatctgggacagattttactttcaccatcagcagcctgcagcctgaagatattgcagtttattactgtcagcaacattataaaactccgtattcgttcggcggagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttag

SEQ ID NO: 13 - DNA, 1389 bp, Artificial Sequence
  Description of Artificial Sequence Synthetic polynucleotide
atgggttggagcctcatcttgctcttccttgtcgctgttgctacgcgtgtccactcccaggtgcagctgcaggagtcgggcccaggactggtgaagccttcggagaccctgtccatcacctgcactgtctctgggttctcattaaccggctatggtatatactgggtgcggcagcccccagggaagggactggagtggctggggatgatatggggtgatggaagaacagactataattcagctctcaaatcccgagtcaccatatcaaaggacaactccaagagccaggtgtccctgaagctgagctctgtgaccgctgcggacacggccaggtattactgtgcgagagattactacggtagtaaggactactggggccagggcaccacggtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaatgtagatcacaagcccagcaacaccaaggtggacaagagagttgagtccaaatatggtcccccatgcccaccatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaatga

(19) United States
(12) Patent Application Publication
(10) Pub. No.: US 2022/0089777 A1
(43) Pub. Date: Mar. 24, 2022

(54) NOVEL HUMANIZED ANTIBODIES AGAINST FACTOR XI HAVING ANTI-THROMBOTIC AND ANTI-INFLAMMATORY EFFECT AND USES THEREOF

(71) Applicants: Aronora, Inc., Portland, OR (US); Oregon Health & Science University, Portland, OR (US)

(72) Inventors: Andras Gruber, Portland, OR (US); Erik I Tucker, Portland, OR (US); Christina U Lorentz, Portland, OR (US)

(21) Appl. No.: 17/424,338

(22) PCT Filed: Jan. 20, 2020
(86) PCT No.: PCT/US2020/014305

§ 371 Date: Jul. 20, 2021

Related U.S. Application Data
(60) Provisional application No. 62/794,987, filed on Jan. 21, 2019.

(51) Int. Cl. C07K 16/36; A61K 39/395; A01N 1/00; A61K 38/48; A61K 31/727; A61K 38/58; A61K 31/4709; A61P 7/02

(52) U.S. Cl. CPC C07K 16/36; A61K 39/3955; A01N 1/00; A61K 38/482; A61K 31/727; A61K 2039/505; A61K 31/4709; A61P 7/02; C07K 2317/24; C07K 2317/92; A61K 38/58

(57) ABSTRACT

Novel binding molecules, comprising humanized antibodies, fragments, variants, and derivatives thereof, compositions, methods, and kits comprising the same, are described, wherein the binding molecules specifically bind to FXI and FXIa. The binding molecules are capable of binding to and forming an immune complex with the factor XI A2 domain, and thereby disrupting the contact activation molecular complex without affecting hemostatic factor XI activity or activation. The binding molecule of this disclosure is useful in safely inhibiting thrombosis and inflammation without compromising hemostasis. The binding molecules, compositions, methods, and kits provided herein are therefore intended to treat, inter alia, thrombosis- and inflammation-related diseases and conditions. Moreover, polynucleotides encoding the binding molecules of this disclosure, vectors comprising the polynucleotides, and host cells for producing the polynucleotides are provided.

### RELATED APPLICATIONS

This application claims the benefit of priority from U.S. provisional patent application No. 62/794,987, filed on 21 Jan. 2019, the disclosure of which is hereby incorporated by reference in its entirety.

### GOVERNMENT SUPPORT

The invention described in this disclosure was supported, at least in part, with United States government grant numbers R44A1088937, R44E1,128016, R43NS077600, and R44HL106919 by the United States Department of Health and Human Services (HHS), National Institutes of Health. The U.S. government has certain rights in inventions arising from this disclosure.

### SEQUENCE LISTING

The instant application contains a Sequence Listing which has been filed electronically in ASCII format and is hereby incorporated by reference in its entirety. The ASCII copy, created on Jan. 15, 2020, is named ARB002PCT_SL.txt and is 18,337 bytes in size.

### FIELD OF INVENTION

This disclosure relates generally to antibodies, and more specifically, to humanized monoclonal antibodies capable of binding to factor XI and methods of use thereof, including methods of use as antithrombotic and anti-inflammatory agents that do not compromise hemostasis.

### BACKGROUND

Thromboembolic disorders, including both venous and arterial thrombosis, are the leading cause of severe chronic morbidity and mortality in developed countries worldwide. These disorders are caused by abnormal blood clot (thrombus) formation that results from accumulation of fibrin, platelets, and other blood cells inside blood vessels, often resulting in blood vessel occlusion, tissue ischemia and, in some circumstances, embolization due to dislodging and migration of clot fragments from the thrombus.

Under normal circumstances blood coagulation results in hemostasis, which is a vital mechanism that prevents blood loss from sites of vascular injury by inducing platelet activation and formation of fibrin. On a mechanistic level, hemostasis proceeds in two simultaneous processes. During primary hemostasis, the blood that comes in contact with the extraluminal environment becomes activated by the essential hemostatic protein, tissue factor (TF), resulting in the instant generation of the coagulation enzyme, thrombin. In turn, thrombin activates other coagulation factors (F), such as fibrinogen (FT), FXIII, FV, and others. Moreover, platelets adhere to the site of trauma and also become activated, predominantly by thrombin, and ultimately aggregate by binding to each other to form a platelet plug. Platelet plug formation is enhanced and stabilized during secondary hemostasis, resulting from a series of continued platelet activation and enzymatic reactions involving coagulation proteins FXI, FIX, FX, FVIII, FVII, FV, FXIII, FII (prothrombin) and FI, which leads to a more stable hemostatic plug that ultimately seals a breach in blood vessel walls, preventing exsanguination and death.

Since 1964, when Macfarlane (Nature. 1964 May 2; 202:498-9) introduced the cascade waterfall model for the process of blood coagulation, the knowledge surrounding the mechanisms and function of blood coagulation in vivo has grown. In the last decades, the theory that two distinct pathways, the so called extrinsic and intrinsic pathways, initiate coagulation and converge into a common pathway that ultimately leads to thrombin generation and fibrin deposition has been partially revised and challenged.

In one model that has generally been accepted by many in the relevant medical field, initiation of thrombin generation during the hemostatic process occurs when the circulating plasma protease activated factor VII (FVIIa) comes into contact and thereby forms a complex with the cofactor, TF. This TF-FVIIa complex converts the zymogens FIX and FX to their active (a) forms FIXa and FXa. FIXa can in turn activate additional FX in the presence of the co-factor, FVIIIa, and FXa can then activate prothrombin (FII) to form thrombin (FIIa) in the presence of the co-factor, FVa, respectively. Thrombin, a key player in coagulation, in turn can catalyze the conversion of fibrinogen into fibrin and cleave the protease activated receptors (PAR) 1 and 4 on platelets, which leads to platelet activation. Activated platelets in combination with fibrin are essential for hemostatic plug formation and therefore are fundamental players of normal hemostasis.

It is well confirmed that FXI is a plasma serine protease zymogen that plays a supporting role in bridging the contact phase and the amplification phase of thrombin generation, in vitro and in vivo (Davie E W et al., Biochemistry. 1991 Oct. 29; 30(43):10363-70, Gailani D and Broze G J Jr., Science. 1991 Aug. 23; 253(5022):909-12; Kravtsov D V et al. Blood. 2009 Jul. 9; 114(2):452-8.). Both physiological hemostatic and pathological prothrombotic thrombin generation involves the activation of FXI and activity of FXIa.

However, data demonstrating that inherited human or other mammalian FXI deficiency usually does not lead to spontaneous bleeding suggest that FXI is not an essential contributor to hemostatic thrombin generation. FXI deficiency, hemophilia C, is associated with an increased risk of bleeding with specific hemostatic challenges, such as certain types of surgical procedures and traumas, although the severity of bleeding correlates poorly with the plasma level or activity of FXI. Meanwhile, severe FXI deficiency in humans has been reported to have certain protective effects from thrombotic diseases, including ischemic stroke and deep venous thrombosis (DVT) (Salomon O et al., Thromb Haemost. 2011 February; 105(2):269-73, Salomon O et al., Blood. 2008 Apr. 15; 111(8):4113-7), and a high level of FXI has been associated with thrombotic events and has been reported to confer higher risk for DVT, myocardial infarction (MI), and stroke (Meijers J C et al., N Engl J Med. 2000 Mar. 9; 342(10):696-701, Berliner J I et al., Thromb Res. 2002 Jul. 15; 107(1-2)55-60, Yang D T et al., Am J Clin Pathol. 2006 September; 126(3):411-5). It has therefore been proposed that pharmacological targeting of FXI may be safer than conventional anticoagulation, and primate studies have provided proof of this hypothesis (Gruber A and Hanson S R, Blood. 2003 Aug. 1; 102(3):953-955, Tucker E I et al., Blood. 2009 Jan. 22; 113(4):936-944).

Taken together, theoretical considerations and previous studies suggest that FXI has a supporting role in maintaining hemostasis but is a significant contributor to the pathogenesis of thrombosis, thereby rendering FXI a promising target for safe antithrombotic therapy. Ample non-clinical and clinical evidence currently supports this concept. Presently available antithrombotic drugs either target the building blocks of thrombi (Fibrin and platelets) or inhibit molecules (coagulation factors) and cells (platelets) from participating in both the thrombus and hemostatic plug forming processes. Antiplatelet, profibrinolytic and anticoagulant agents have been the mainstay for the treatment and prevention of thromboembolic diseases for decades and are among the most commonly prescribed drugs in clinical practice. Yet, most of these agents can completely block both thrombosis and hemostasis when administered in effective doses, and therefore have dose-limiting antihemostatic toxicity. As a result, current antithrombotic agents are administered by medical practitioners at lower than fully effective doses in a careful attempt to balance the antithrombotic benefit with the potential for severe and even fatal hemorrhage.

So far, one of the few examples for an anti-FXI antibody exhibiting therapeutic potential is murine antibody 1A6 (also named aximab) as published by Tucker et al. (Prevention of vascular graft occlusion and thrombus-associated thrombin generation by inhibition of factor XI. Erik I. Tucker, Ulla M. Marzec, Tara C. White, Sawan Hurst, Sandra Rugonyi, Owen J. T. McCarty, David Gailani, András Gruber, and Stephen R. Hanson. Blood. 2009 Jan. 22; 113(4):936-944). Antibody 1A6 is also disclosed in U.S. Pat. No. 9,125,895, which is incorporated herein by reference in its entirety. However, as antibody 1A6 is a murine antibody, it is unsuitable for human therapies especially for chronic applications such as antithrombotic therapy. Likewise, another example for an anti-FXI antibody exhibiting therapeutic potential is a differentially acting murine antibody 14E11 (also named xisomab) as published by Cheng et al. (A role for factor Mk-mediated factor XI activation in thrombus formation in vivo, Cheng Q I, Tucker E I, Pine M S, Sisler I, Matafonov A, Sun M F, White-Adams T C, Smith S A, Hanson S R, McCarty O J, Renné T, Gruber A, Gailani D. Blood. 2010 Nov. 11; 116(19):3981-3989; Luo, D., et al. (2012) Infect Immun. 80(1):9109; Tucker. E., et al. (2012) Blood. 119(20):4762-8). Antibody 14E11 is also disclosed in U.S. Pat. Nos. 9,637,550, 8,940,883, and 8,388,959 (“14E11 Patents”).

One method to convert a murine antibody into an acceptable therapeutic antibody is humanization. Standard techniques are available to a person skilled in the art such as those described in O'Brien S. and Jones T. (2001. Humanising Antibodies by CDR Grafting. In: Kontermann R., Dübel S. (Eds) Antibody Engineering. Pp. 567-590. Springer Lab Manuals. Springer, Berlin, Heidelberg), in Hwang, Almagro, Buss, Tan, and Foote (2005) Use of human germline genes in a CDR homology-based approach to antibody humanization. Methods, 36(1):35-42), and in the references cited therein. For further reduction of the inherent immunogenicity potential of humanized antibodies, further sequence optimization and germlining may be required.

These standard methods were applied to humanize and optimize the murine 14E11 antibody, the resulting recombinant humanized antibody, hereinafter referred to AB023, exhibited binding activity in a biochemical assay comparable to the murine precursor. By introduction of sequence alterations, humanized variants of murine antibody 14E11 have been generated, e.g., antibody AB023, which display both comparable biochemical profiles and anti-thrombotic efficacy in vivo.

Cardiovascular disease and venous thromboembolism (VTE) remain leading causes of death. Primary prevention, acute treatment, and secondary preventative strategies such as anticoagulation and anti-platelet therapy are effective, but universally increase the risk of bleeding. Thus, this disclosure fulfills the pressing medical need for safe yet efficacious agents for antithrombotic and anti-inflammatory therapy treatment without paralyzing hemostasis.

### SUMMARY

The invention described in this disclosure overcomes the existing drawbacks and prior art by providing binding molecules, compositions, methods, and kits for inhibiting thrombosis without compromising hemostasis. Compositions of this disclosure include recombinant, humanized anti-FXI apple 2 domain binding molecules, binding fragments thereof, variants thereof, derivatives thereof, cell lines, and nucleic acid molecules encoding the amino acid sequences of the binding molecules. The disclosure further comprises pharmaceutical compositions comprising a therapeutically effective amount of the binding molecules, binding fragments, variants, and derivatives thereof, in a pharmaceutically acceptable carrier and methods of use thereof. Methods of this disclosure may comprise administration of the compositions of this disclosure to a subject in need thereof for the purpose of inhibiting thrombosis, preventing thrombosis, or treating inflammation via, e.g., antithrombotic and anti-inflammatory activity by blocking factor XIIa-mediated FXI activation without inhibiting FXI activation by thrombin or the procoagulant function of FXIa. Methods for making the binding molecules, binding fragments, variants, and derivatives thereof, are also provided.

In a preferred aspect, this disclosure provides a binding molecule comprising:

a CDR1 of the light chain comprising the sequence KASQDVSTAVA (SEQ ID NO: 1);
a CDR2 of the light chain comprising the sequence LTSYRNT (SEQ ID NO: 2);
a CDR3 of the tight chain comprising the sequence QQHYKTPYS (SEQ ID NO: 3);
a CDR1 of the heavy chain comprising the sequence GYGIY (SEQ ID NO: 4);
a CDR2 of the heavy chain comprising the sequence MIWGDGRTDYNSALKS (SEQ ID NO: 5); and
a CDR3 of the heavy chain comprising the sequence DYYGSKDY (SEQ ID NO: 6).

The binding molecule may comprise a V_{H} region as depicted in SEQ ID NO: 8 and/or a V_{L} region as depicted in SEQ ID NO: 9.

The binding molecule may comprise a light chain as depicted in SEQ ID NO: 10 or encoded by SEQ ID NO: 12 and/or a heavy chain as depicted in SEQ ID NO: 11 or encoded by SEQ ID NO: 1.3.

The binding molecule of this disclosure is capable of binding to mammalian FXI and/or FXIa, including to a human or non-human primate FXI or a human or non-human primate FXIa.

Specifically, the binding molecule is capable of binding to and forming a therapeutic immune complex within an amino acid sequence corresponding to the A2 domain of FXI comprising amino acids 91-175 of SEQ ID NO: 7. Here, numbering of the amino acids of human FXI includes the signal sequence staring with the methionine at position −18 to −1 and then starting with the glutamine at position 1. It is contemplated that the binding molecule may be an antibody, an antigen-binding fragment, a variant, or a derivative thereof, and specifically a humanized monoclonal antibody, an antigen-binding fragment, a variant, or a derivative thereof, for example an IgG antibody.

In a further aspect, this disclosure provides a polynucleotide encoding a binding molecule as defined herein, and a vector, e.g., an expression vector, comprising the polynucleotide. The disclosure also relates to a host cell comprising the vector or polynucleotide.

In a further aspect, a process for the production of a binding molecule as described herein is provided, the process comprising culturing a host cell, as defined herein, under conditions allowing the expression of the binding molecule and optionally recovering the produced binding molecule from the culture.

Moreover, the disclosure relates to a pharmaceutical composition comprising a binding molecule, polynucleotide, the vector and/or the host cell, as defined herein, and optionally one or more pharmaceutically acceptable excipients. The pharmaceutical composition may comprise one or more additional active agents, e.g., anti-thrombotic and/or anticoagulant agents, or may be administered as part of combination therapy with additional active agents.

According to this disclosure, a binding molecule, polynucleotide, vector, host cell, or pharmaceutical composition can be used in a method of inhibiting contact activation, blood coagulation, platelet aggregation, and/or thrombosis in a subject, and are therefore useful in the treatment and/or prophylaxis of disorders, e.g., cardiovascular, infectious, or inflammatory disorders, preferably thrombotic or thromboembolic disorders and/or thrombotic or thromboembolic complications.

Further provided herein is the use of the binding molecule as an anticoagulant in blood samples, blood preservations, plasma products, biological samples, or medicinal additives or as a coating on medical devices.

Moreover, this disclosure relates to a kit comprising a binding molecule, polynucleotide, vector, host cell, or the pharmaceutical composition, as described herein.

### DESCRIPTION OF THE FIGURES

The accompanying drawings are incorporated into and form a part of the specification to illustrate the present disclosure. These drawings, together with the description, explain the principles of the disclosure. The drawings illustrate preferred and alternative examples and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 is a graph showing the concentration-dependent effect of 14E11 (10⁻⁵ to 10⁰ μM) on the activated partial thromboplastin time (aPTT) in mouse (open circles) and human (black circles) plasma.

FIGS. 2A-F are blots and graphs demonstrating the binding properties of 14E11. FIG. 2A depicts Coomassie blue stained 10%-polyacrylmide gel of human (H) and mouse (M) recombinant FXI; FIGS. 2B and C show Western blots of non-reducing 10% polyacrylamide gels of mouse (B) and human (C) normal (N) and FXI deficient (XI−/−) plasmas, using biotinylated-14E11 for detection. rXI in panel B indicates recombinant mouse FXI control; FIG. 2D shows binding of biotinylated-14E11 to immobilized mouse FXI (open circles), human FXI (closed circles) or human FXIa (open squares); FIG. 2E shows a western blot of non-reducing 10% polyacrylamide gel of human FXI (hXI), human prekallikrein (PK), and human FXI in which the A1, A2, A3, or A4 domain has been replaced with the corresponding domain from PK. Position for FXI dimer is indicated to the right by “D”, and for monomeric PK by “M” (note FXI with the PK A4 domain is a monomer because A4 mediates FXI dimer formation); and FIG. 2F shows a western blot (left panel) of a non-reducing 10% polyacrylamide gel of human FXI (hXI) and individual human FXI apple domains linked to tissue plasminogen activator (tPA) and the right panel is a stained gel showing the recombinant apple domain-tPA chimeras (note the A4 chimera forms a dimer). For panels A-C and E, positions of molecular mass standards in kDa are at the left of the figures, and for panel F at the right.

FIGS. 3A-B are graphs showing the effects of 14E11 (closed circles) and the humanized version, AB023 (open circles), on in vitro aPTT. FIG. 3A shows the effects of 14E11 and AB023 in pooled human plasma; and FIG. 3B shows the effects of 14E11 and AB023 in pooled baboon plasma. Both 14E11 and AB023 similarly prolong aPTT in both human and baboon plasma.

FIGS. 4A-F are blots and graphs demonstrating the binding properties of AB023. FIG. 4A shows western blot of non-reducing 10% polyacrylamide gel of human FXI (lane 1) and human FXI in which the A1, A2, A3, or A4 domain has been replaced with the corresponding domain from prekallikrein (PK), biotinylated AB023 was used for detection; FIG. 4B shows a western blot of non-reducing 10% polyacrylamide gel of individual human FXI apple domains (A1-A4) linked to recombinant tPA where AB023 recognizes the A2 domain of human FXI; FIG. 4C shows binding of AB023 to human FXI (closed circles), human FXI a (open square) and mouse FXI (closed triangles): FIG. 4D shows AB023 concentration-dependently inhibits FXIIa activation of FXI; FIG. 4E shows AB023 does not prevent activation of FXI by thrombin; and, FIG. 4F shows that AB023 prolongs the aPTT in a concentration dependent manner in plasma from human (closed circles), baboons (open squares), cynomolgus monkeys (open circles) and rats (open diamonds). *The FXI/PKA4 chimeric protein (A4*) is a dimeric molecule created by replacing Cys326 with alanine in PK A4 domain.

FIG. 5 shows the effect of AB023-FXI complex formation on in vitro aPTT. These experiments were performed in sequential order using FXI deficient plasma (George King, Product #1100, Lot #6538). Baseline aPTT in FXI deficient plasma was determined to be 118.5 s (circles). Recombinant human FXI (Enzyme Research Labs, Cat. #HFXI1111) was added to the FXI deficient plasma for a final concentration of 10 μg/mL and aPTT was measured. Addition of FXI to FXI deficient plasma shortened the aPTT to 32.3 s (squares). Subsequently, AB023 was added to the FXI deficient plasma+10 μg/mL FXI mixture at a final antibody concentration of 100 μg/mL and aPTT was prolonged to 66.6 s (triangles). In a second experiment, AB023 was added to the FXI deficient plasma for a final antibody concentration of 100 μg/mL and aPTT was measured. Addition of AB023 to FXI deficient plasma did not change the aPTT from baseline (117.7 s, inverted triangles). Addition of recombinant human FXI (final concentration 10 μg/mL) to this mixture shortened the aPTT to 59.1 s (diamonds), similar to that seen in the previous experiment. These data demonstrate that the anticoagulant effect of AB023 only occurs when a complex between FXI and AB023 is generated.

FIG. 6 shows the effect of 14E11 and AB023 in a murine model of experimental arterial thrombosis. C57B1/6 mice (treated with or without, 14E11 (1.0 mg/kg, i.v.), or AB023 (1.0 mg/kg, i.v.)) or FXI−/−mice were tested in a FeCl₃ carotid artery thrombosis model. FeCl₃ concentrations from 2.5% to 10% were applied to the carotid artery and time to occlusion was measured. Bar heights indicate the percent of mice in each group with patent arteries 30 minutes after applying FeCl₃. Intravenous infusion of 1.0 mg/kg AB023 into wild type mice (bars with circles) protected mice from carotid occlusion induced by 3.5%, 5.0% and 7.5% FeCl₃ compared to wild type mice not treated (bars with dots). These results are comparable to those from FXI−/− mice (open bars) and mice treated with 14E11 (checkered bars), (n=10/group).

FIG. 7. shows the relationship of AB023 plasma concentrations and aPTT in four baboons. Each graph represents the time course of AB023 plasma concentration (left y-axes, closed circles) and aPTT (right y-axes, open circles) in a single baboon that was administered 1.0 mg/kg of AB023, i.v. In each baboon, aPTT was prolonged until plasma AB023 concentrations fell below detectable levels (1000 ng/mL), using a partially validated ELISA assay to detect free AB023 in baboon plasma. aPTT is shown as fold change over baseline.

FIGS. 8A-D show the effect of AB023 in an in vivo baboon thrombosis model (graft expansion chamber). AB023 reduces platelet-rich thrombus growth in a primate thrombosis model. Effect of AB023 (0.2 mg/kg, i.v) on platelet (as shown in FIGS. 8A and 8C) deposition on collagen-coated (4 mm diameter, 2 cm long) vascular grafts (FIG. 8A) and venous expansion chamber (9 mm diameter, 2 cm long) (FIG. 8C). FIGS. 8B and 8D depict fibrin deposition within the collagen graft (FIG. 8B), venous expansion chamber (FIG. 8D) in control treated (hatched bar) and after AB023 treatment (open bar). Values are mean±SEM, n=7 experiments/group in 4 animals for control group (includes historical controls from the same experiment performed using 14E11), n=2 experiments in 2 animals for the AB023 treatment. Values are mean±SEM.

FIGS. 9A-F show the effect of AB023 on platelet-rich thrombus growth in an in vivo baboon thrombosis model (collagen-coated graft). FIGS. 9A-9C show the effect of AB023 (1.0 mg/kg, i.v) on platelet (FIGS. 9A-C) deposition on collagen-coated (4 mm diameter, 2 cm long) vascular grafts (FIG. 9A) and 10 cm downstream of the collagen-coated graft (“tail”) (FIG. 9B); FIG. 9C depicts platelet deposition in both the graft+tail combined; FIGS. 9D-F depict fibrin deposition within the collagen graft (FIG. 9D), tail (FIG. 9E), and graft+tail combined (FIG. 9F) in control treated (hatched bar) and after AB023 treatment. Values are mean±SEM, n=4 experiments/group in 4 animals. *p<0.05, **p<0.01, ***p<0.001 vs. control. Each animal underwent a control experiment followed by an AB023 experiment.

FIGS. 10A-D show a comparison of the activity of AB023 with that of the murine antibody 14E11. FIGS. 10A-B show the effects of 14E11 and AB023 on FXI autoactivation in presence of dextran sulfate (FIG. 10A) and DNA (FIG. 10B) as a function of 14E11 (dotted bars) and AB023 (hatched bars) antibody concentration The humanized antibody AB023 inhibits the DNA-induced autoactivation of purified human FXI in a concentration-dependent manner, in vitro; FIG. 10C) shows the effects of 14E11 (open circles) and AB023 (black squares) on inhibition of FXIIa activation of FXI compared to control (black circles); and FIG. 10D) shows the effects of 14E11 and AB023 on FXII activation by FXIa in vitro. The graph depicts FXIIa activity is shown as a function of antibody concentration. A mixture of purified human FXII and FXIa was incubated with varying concentrations of 14E11 (dotted bars) or AB023 (hatched bars) and FXIIa amidolytic activity was measured. AB023 inhibits activation of purified human FXII by purified human FXIa in a concentration-dependent manner, in vitro, whereas 14E11 does not.

### DETAILED DESCRIPTION

Reference will now be made in detail to representative embodiments of this disclosure. While the antibody, fragments, variants, and derivatives thereof disclosed will be described in conjunction with the enumerated embodiments, it will be understood that they are not intended to limit this disclosure to those embodiments. On the contrary, the antibody, fragments, variants, and derivatives thereof disclosed are intended to cover all alternatives, modifications, and equivalents that may be included within the scope of this disclosure as defined by the claims. One skilled in the art will recognize many methods and materials similar or equivalent to those described herein, which could be used in and are within the scope of the practice of this disclosure. This disclosure is in no way limited to the methods and materials described.

All publications and patents mentioned herein are incorporated herein by reference in their respective entireties for the purpose of describing and disclosing, for example, the constructs and methodologies that are described in the publications which might be used in connection with this disclosure. The publications discussed throughout the text are provided for their disclosure prior to the filing date of the present application. Nothing herein is to be construed as an admission.

Unless defined otherwise, technical and scientific terms used herein have the same meaning as commonly understood by one of ordinary skill in the art to which this disclosure belongs. The term “a” or “an” refers to one or more of the entity to which it refers, and thus, is understood to mean, for example, “one or more” and “at least one” which may be used interchangeably.

The practice of this disclosure employs, unless otherwise indicated, conventional techniques of cell biology, cell culture, molecule biology, transgenic biology, microbiology, recombinant DNA, and immunology, which are within the skill of the art. Such techniques may be found set forth fully in the literature, see, for example, Sambrook et al., ed. (1989) Molecular Cloning A Laboratory Manual (2^{nd} ed.; Cold Spring Harbor Laboratory Press); Sambrook et al., ed. (1992) Molecular Cloning: A laboratory Manual, (Cold Spring Harbor Laboratory, NY); D. N. Glover ed., (1985) DNA Cloning, Volumes I and II, Gait, ed. (1984) Oligonucleotide Synthesis; Mullis et al. U.S. Pat. No. 4,683,195; Flames and Higgins, eds. (1984) Nucleic Acid Hybridization; Hames and Higgins, eds. (1984) Transcription and Translation; Freshney (1987) Culture of Animal Cells (Alan R. Liss, Inc.; Immobilized Cells and Enzymes (IRL Press (1986); Perbal (1984) A Practical Guide to Molecular Cloning; the treatise, Methods in Enzymology (academic Press. Inc., N.Y.) Miller and Calos eds. (1987) Gene Transfer Vectors for Mammalian Cells, (Cold Spring Harbor Laboratory); Wu et al., eds., Methods in Enzymology, Vols. 154 and 155; Mayer and Walker, eds. (1987) Immunochemical Methods in Cell and Molecular Biology (Academic Press, London; Weir and Blackwell, eds., (1986) Handbook of Experimental Immunology, Volumes I-IV; Manipulating the Mouse Embryo, Cold Spring Harbor Laboratory Press, Cold Spring Harbor, N. S., (1986); and in Ausubel et al. (1989) Current Protocols in Molecular Biology (John Wiley and Sons. Baltimore, Md.). Although any methods, devices, and materials similar or equivalent to those described herein can be used in the practice or testing of the antibody, fragments, variants, and derivatives thereof disclosed, the preferred methods, devices and materials are now described.

Inhibition of coagulation factor XI (a.k.a., FXI)—which is ascribed a role in development of pathological thrombus formation, while having limited effect on physiologic hemostasis—is a promising novel approach in the development of new anti-thrombotic agents to achieve an improved benefit-risk ratio. This disclosure, infer alia, provides a novel binding molecule, AB023, that is capable of specifically binding to FXI, forming the immune complex FXI-AB023, and thereby inhibiting the proper molecular assembly of a normally functioning contact activation complex comprising FXII, FXI, prekallikrein (PK), and high molecular weight kininogen (HMWK). As a result, the molecular interactions between FXI-AB023. FXII PK and HMWK are limited, and therefore conversion of FXI-AB023 into its activated form FXIa-AB023 by FXIIa, and conversion of FXII into its activated form FXIIa by FXIa-AB023 are also limited. The binding molecule AB023 is a novel anticoagulant recombinant monoclonal antibody directed against FXI. Moreover, the binding molecule has also been shown to bind to FXIa. The binding molecule provided herein therefore blocks the reciprocal activation of the players involved in pathological thrombin generation and thrombosis, including kallikrein and bradykinin generation that are involved in blood pressure regulation and inflammation (reviewed by Weidmann, H., et al. (2017) Biochim Biophys Acta Mol Cell Res. 1864(11 Pt B):2118-2127. Björkvist et al., (2014) Thrombosis and Hemostasis. 112(5):868-75; Blood Advances 2019 3:658-669). Specifically, a humanized version of the murine 14E11 monoclonal antibody that advantageously binds to FXI with a high binding affinity comparable to 14E11 is provided. Moreover, the immune complex formation between the binding molecule and FXI effectively reduces blood clotting in vitro, as indicated by prolongation of the activated partial thromboplastin time (aPTT) in the presence of such complexes that form at low concentrations of the binding molecule. The binding molecule of this disclosure is therefore a promising new agent for effective treatment and/or prophylaxis of disorders where activation of the contact system plays a pathogenic role, especially in inflamatory and thrombotic or thromboembolic disorders and/or thrombotic or thromboembolic complications, and is moreover thought to be effective without severely compromising hemostasis, thereby minimizing the risk of bleeding.

With the antibody of this disclosure, a therapeutic molecule has been generated which reduces immunogenicity risk and, after forming an immune complex with circulating FXI, reduces thrombus development in vivo. The formation of this immune complex between the antibody and the free FXI antigen in vivo effectively blocks thrombus propagation, but without compromising hemostasis. Indeed, formation of the immune complex between the humanized 14E11 antibody, AB023, and FXI does not interfere with the hemostatic feedback activation of the FXI-AB023 immune complex by thrombin. Moreover, the FXIa-AB023 immune complex retains its enzymatic activity that contributes to hemostatic thrombin generation through activation of FIX and other coagulation factors by FXIa, thereby making antithrombotic therapy by the antibody, binding fragments, variants, and derivatives thereof, of this disclosure hemostatically safer than directly inhibiting the enzymatic activity or hemostatic activation of FXI, and thus broadening the range of clinical indications and scenarios in which this type of antithrombotic therapy can be applied. It is of importance to note that in the absence of circulating immune complex, the antibody alone has no anticoagulant or antithrombotic activity. In addition, in the absence of free, available, and activatable FXI in the circulation, the antibody alone has no anticoagulant or antithrombotic or other activities. Thus, in the absence of circulating FXI-AB023 immune complexes, the antibody of this disclosure may lack anticoagulant activity, and may have no antithrombotic activity in FXI deficient subjects.

### Binding Molecule

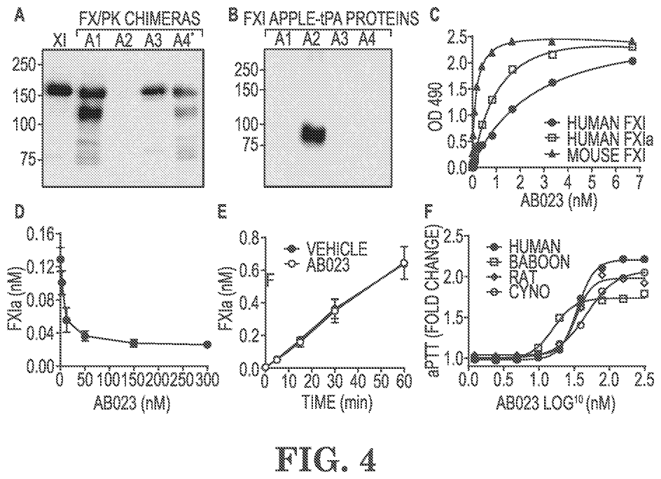

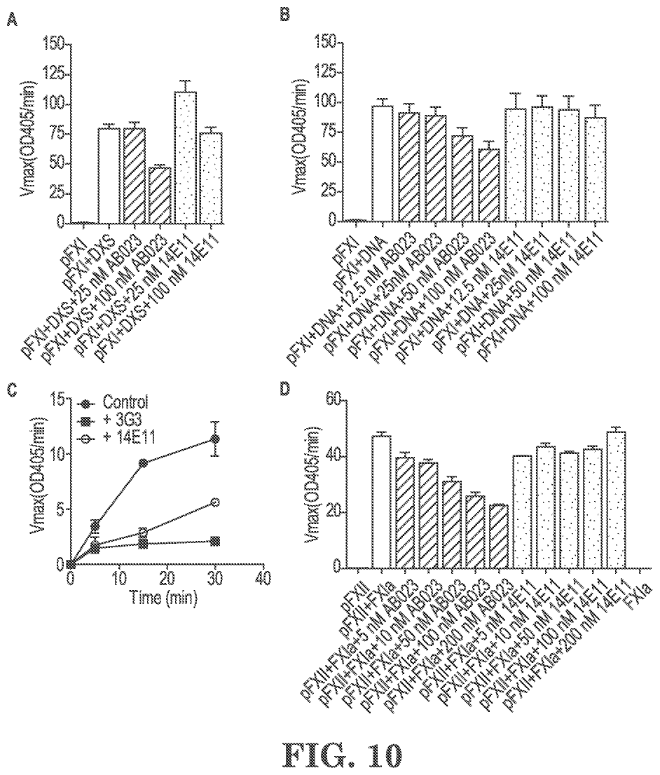

The binding molecule of this disclosure is a novel anticoagulant recombinant monoclonal antibody directed against coagulation factor XI (FXI). It was obtained by humanization using complementarity determining region (CDR)-grafting of the mouse monoclonal antibody 14E11 disclosed in U.S. Pat. Nos. 8,388,959, 8,940,883 and 9,637,550 entitled: Anti-FXI Antibodies and Methods of Use. Surprisingly, the binding molecule of this disclosure does not comprise a number of amino acid substitutions in the CDR regions as compared to the 14E11 CDRs and yet exhibits advantageous properties. The murine monoclonal antibody 14E11, and the humanized antibody, A13023, were characterized and their anticoagulant properties assessed both in vitro and in vivo in order to show performance comparability. The binding molecule of this disclosure is capable of binding to FXI with binding affinity comparable to 14E11, and FXI in the immune complex is not efficiently converted into FXIa by FXIIa, while it is efficiently converted into FXIa by thrombin (FIG. 4). Interestingly, it appears that AB023 is more potent at inhibiting this activation than 14E11 (FIG. 10C). The FXIa-AB023 complex has reduced catalytic activity for converting MI into its active form, FXIIa, in contrast to 14E11 (FIG. 10D). If FXI-AB023 is converted by thrombin into FXIa AB023, it retains enzymatic activity towards FIX (data not shown), and other macromolecular substrates and small molecule substrates. As a result, contact activation-mediated events are downregulated while hemostatic thrombin mediated activation of FXI-AB023 preserves the hemostatic activity of circulating FXI.

The binding molecule of this disclosure is a humanized monoclonal antibody, antigen-binding fragment, variant, or derivative thereof, and preferably, AB023, monoclonal therapeutic antibody that targets FXI. It may be an IgG4 and may have a S241P hinge modification to prevent antibody arm exchange. The amino acid sequence of the light (LC) chains is shown in SEQ ID NO: 10 and the encoding DNA sequence for the LC is shown in SEQ ID NO: 12. The amino acid sequence of the heavy (HC) chains is shown in SEQ ID NO: 11 and the encoding DNA sequence for the HC is shown in SEQ ID NO: 13. AB023 was generated by CDR grafting and contains a kappa (κ) light chain and an IgG4 isotype heavy chain. The variable sequences (VH and VL) from the murine monoclonal precursor antibody, 14E11, were cloned into a human IgG4 (SP241 hinge modified, using the Kabat numbering system) heavy chain gene and a kappa light chain gene. The four chains are held together by a combination of covalent (disulfide) and non-covalent bonds. There are 16 cysteine residues, and accordingly, [16/2] potential disulfide bonds per molecule. The heavy chain subunit contains one consensus sequence (N-X-S/T) for potential N-linked glycosylation located on the heavy chain.

The antithrombotic effects seen with 14E11 were maintained after humanization, and AB023 prevented venous-type and arterial-type thrombosis. This disclosure, in a first aspect, relates to a binding molecule capable of specifically binding to factor XI, in which the binding molecule comprises the following complementarity determining regions (CDRs): CDR1 of the light chain comprising the sequence KASQDVSTAVA (SEQ ID NO: 1); CDR2 of the light chain comprising the sequence LTSYRNT (SEQ ID NO: 2); CDR3 of the light chain comprising the sequence QQHYKTPYS (SEQ ID NO: 3); CDR1 of the heavy chain comprising the sequence GYGIY (SEQ ID NO: 4); CDR2 of the heavy chain comprising the sequence MIWGDGRTDYNSALKS (SEQ ID NO: 5); and CDR3 of the heavy chain comprising the sequence DYYGSKDY (SEQ ID NO: 6). The binding molecule may further comprise a S241P modification

During the humanization process, the CDR regions of 14E11 were determined and the variable regions of both the VH and VL were plugged into a modeling program to identify which amino acid residues in the framework were useful for the binding properties of the antibody. The CDR regions were then grafted onto a human framework that had the highest degree of homology with the 14E11 framework. If needed, back mutation to specific murine framework identified to be useful for binding. From this process, 3VH and 3VL were generated. In some embodiments, the antibody AB023 is a combination of VH3 (SEQ. ID NO. 8) and VL3 (SEQ. ID NO. 9).

The term “amino acid” or “amino acid residue” refers to an amino acid having its art recognized definition such as an amino acid selected from the group consisting of: alanine (Ala or A); arginine (Arg or R); asparagine (Asn or N); aspartic acid (Asp or D); cysteine (Cys or C); glutamine (Gln or Q); glutamic acid (Glu or E); glycine (Gly or G); histidine (His or H); isoleucine (He or I): leucine (Leu or L); lysine (Lys or K), methionine (Met or M); phenylalanine (Phe or F), proline (Pro or P), serine (Ser or S); threonine (Thr or T); tryptophan (Trp or W); tyrosine (Tyr or Y); and valine (Val or V), although modified, synthetic, or rare amino acids may be used as desired. Generally, amino acids can be grouped as having a nonpolar side chain (e.g., Ala, Cys, He, Leu, Met, Phe, Pro, Val); a negatively charged side chain (e.g., Asp, Glu); a positively charged sidechain (e.g., Arg, His, Lys); or an uncharged polar side chain (e.g., Asn, Cys, Gln, Gly, His, Met, Phe, Ser, Thr, Trp, and Tyr).

### Number and Distribution of Substitutions

The amino acid substitutions can generally be distributed across the CDRs in any way, i.e., one CDR may for instance comprise one or more exchanges, and a second CDR may comprise one or more substitutions. Or two CDRs may comprise one or more amino acid substitutions, or all six CDRs may comprise amino acid substitutions, e.g., one or two substitutions per CDR, and preferably, a binding molecule comprising one or more substitutions in the CDR1, CDR2 and/or CDR3 of the light chain or one or more amino acid substitutions in CDR1, CDR2 and/or CDR3 of the heavy chain, that retain—to the greatest extent possible without destroying functionality—the CDRs of the non-humanized molecule. Generally, the amino acid substitutions can be distributed virtually in any manner, as long as the number of cumulative amino acid substitutions as compared to the 14E11 CDR amino acid does not abolish the binding molecule's capability to bind to FXI.

### Type of Substitutions

In general, any combination of amino acid substitutions in the CDRs as compared to 14E11 is conceivable as long as it does not abolish the advantageous properties of the binding molecules of this disclosure. Amino acid exchanges can be conservative (i.e., exchanging an amino acid of one class or group for another amino acid from the same class or group as listed above) or non-conservative (i.e. exchanging an amino acid from one class/group for another amino acid from another class/group).

Preferred substitutions yield binding molecules of this disclosure which lead to prolongation of the aPTT, as described herein.

Binding molecules according to this disclosure may comprise one or more of the aforementioned CDRs, optionally in combination. Preferred substitutions yield binding molecules of this disclosure which lead to about a 1.5-fold, 2-fold, or higher prolongation of the aPTT as described herein.

A preferred binding molecule according of this disclosure, which may be a monoclonal antibody, antigen-binding fragment, variant, or derivative thereof, comprises the following CDRs: a CDR1 of the light chain comprising the sequence KASQDVSTAVA (SEQ ID NO: 1); a CDR2 of the light chain comprising the sequence LTSYRNT (SEQ ID NO: 2); a CDR3 of the light chain comprising the sequence QQHYKTPYS (SEQ ID NO: 3); a CDR1 of the heavy chain comprising the sequence GYGIY (SEQ ID NO: 4); a CDR2 of the heavy chain comprising the sequence MIWGDGRTDYNSALKS (SEQ ID NO: 5); and a CDR3 of the heavy chain comprising the sequence DYYGSKDY (SEQ ID NO: 6). The preferred binding molecule may further and optionally comprise a S241P hinge modification.

Moreover, binding molecules of this disclosure are envisaged to comprise a variable region of the light chain (V_{H} or VH region) as depicted in SEQ ID NO: 8, and/or a variable region of the heavy chain (V_{L}, or VL region) as depicted in SEQ ID NO: 9. However, other combinations of V_{L} and V_{H} regions are also conceivable. Accordingly, a preferred embodiment is humanized monoclonal antibody AB023 with sequences as disclosed herein and depicted in SEQ ID NOS: 1, 2, 3, 4, 5, 6, 8, and 9.

### Factor XI

As set out herein, the binding molecule of this disclosure is preferably capable of binding to two identical exosites on the FXI homodimer that participate in select macromolecular substrate recognition reactions. Human “factor XI”, which may also be referred to herein as “plasma thromboplastin antecedent”, “PTA”. “Rosenthal factor”, “coagulation factor XI”, “FXI,” “FII”, or “fXI,” circulates in blood as a two-chain glycoprotein homodimer with a combined molecular weight of approximately 160 kilo Daltons (kD). The two monomers that form the homodimer are identical disulfide bonded polypeptides with molecular weights of approximately 80,000 daltons each. Each FXI monomer contains 4 “apple domains” (A1 to A4 from the N-terminus, heavy chain of the monomer) and a C-terminal catalytic domain (light chain of the monomer). Without wishing to be bound by specific theory, it is thought by some experts in the field that the 4 apple domains contain the FXI binding sites for other proteins, such as A1 for thrombin; A2 for high molecular weight kininogen (HK, HMWK), A3 for FIX, glycoprotein Ib (GPIb), and heparin, and A4 for dimerization and possibly for FXIIa. FXI can be converted into its active form, the coagulation FXIa by FXIIa, by thrombin. FXIa, and possibly other proteases. The serine protease FXIa can cleave a number of macromolecular substrates, including FXII, FX FV, TFPI, FIX, and possibly others. One of the best described reactions is the common aPTT assay that is sensitive to the conversion of FIX into FIXa, and it can be easily measured in plasma or blood in regular clinical laboratories. FXIa subsequently activates coagulation factor IX (IXa), which can activate coagulation factor X (FXa), which then can mediate coagulation FII (prothrombin) activation into thrombin. Thrombin then can activate additional FXI molecules, thereby amplifying the enzymatic process through a positive feedback reaction, which then leads to the generation of more thrombin, and consequential coagulation of recalcified citrated blood or plasma in the aPTT assay in usually less than 40 seconds from initiation of the reaction with negatively charged surfaces and phospholipids.

The term “Factor XI” refers to the human coagulation factor XI (FII, FXI) with Uniprot Acc. No. P03951, entry version 194 of 14 Oct. 2015 (SEQ ID NO: 7). As set out elsewhere herein, the binding molecule of this disclosure is envisaged to bind to a domain within an amino acid sequence corresponding to amino acids 91-175 of SEQ ID NO: 7. Numbering of the amino acids of human FXI includes the signal sequence starting with methionine at position −18 to −1 and then starting with the glutamine at position 1.

The term “position” when used in accordance with the disclosure means the position of either an amino acid within an amino acid sequence depicted herein or the position of a nucleotide within a nucleic acid sequence depicted herein. The term “corresponding” as used herein also includes that a position is not only determined by the number of the preceding nucleotides/amino acids, but is rather to be viewed in the context of the circumjacent portion of the sequence. Accordingly, the position of a given amino acid or nucleotide in accordance with the disclosure may vary due to deletion or addition of amino acids or nucleotides elsewhere in the sequence. Thus, when a position is referred to as a “corresponding position” in accordance with the disclosure it is understood that nucleotides/amino acids may differ in terms of the specified numeral but may still have similar neighboring nucleotides/amino acids. In order to determine whether an amino acid residue (or nucleotide) in a given sequence corresponds to a certain position in the amino acid sequence (or polynucleotide sequence) of a “parent” amino acid (or polynucleotide sequence) (e.g., that of human FXI as depicted in SEQ ID NO: 7), the skilled person can use means and methods well-known in the art, e.g., sequence alignments, either manually or by using computer programs as exemplified herein.

The term “epitope” in general refers to a site on an antigen, i.e., a (poly-) peptide, which a binding domain recognizes, and can also be referred to as an “antigenic structure” or “antigenic determinant”. The term “binding domain” refers to an “antigen binding site”, i.e., characterizes a domain of a binding molecule which hinds/interacts with a given target epitope on an antigen or a group of antigens, e.g., the identical antigen in different species. A target antigen may comprise a single epitope, and preferably comprises at least two epitopes, and can include any number of epitopes, depending on the size, conformation, and type of antigen. Furthermore, it should be noted that an “epitope” on a target antigen may be a target (poly) peptide, but may also be or include non-polypeptide elements, e.g., an epitope may include a carbohydrate side chain. The term “epitope” in general encompasses linear epitopes and conformational epitopes. Linear epitopes are contiguous epitopes comprised in the amino acid primary sequence and may, e.g., include at least 2 amino acids or more. Conformational epitopes are formed by non-contiguous amino acids juxtaposed by folding of the target antigen, and preferably target (poly-)peptide.

Binding molecules of this disclosure are envisaged to recognize a structurally conserved epitope located on the heavy chain of factor XI that contains a sequence of at least 2, at least 3, at least 4, at least 5, at least 6, at least 7, at least 8, at least 9, at least 10 contiguous or noncontiguous amino acids of factor XI (SEQ ID NO: 7).

The binding molecules provided herein bind to and form an immune complex with the A2 domain of human factor XI which comprises amino acids 91-175 of SEQ ID NO: 7. However, it is also envisaged that the binding molecule is capable of binding to variants of human FXI as specified herein, as the parent molecule of AB023, 14E11 forms immune complex or complexes with FXI in plasmas from multiple but not all mammalian animal species. If the binding molecule is in the native (IgG4) form, it can bind one or two FXI homodimers, and multimers or aggregates may form as well. The term “variant” when used in relation to FXI refers to a polypeptide comprising one or more amino acid sequence substitutions, deletions, and/or additions as compared to a “parent” FXI sequence and exerts the same biologic function, i.e., can be converted to its active form FXIa, which has protease activity and catalyzes the activation of FIX and/or activation/inactivation of other macromolecular substrates, such as TFPI, SERPIN-s, protein S, FV, FX, and FXII. Amino acid substitutions may be conservative, as defined herein, or non-conservative or any combination thereof. FXI variants may have additions of amino acid residues either at the carboxy terminus or at the amino terminus (where the amino terminus may or may not comprise a leader sequence). The term “variant” when used in relation to FXI includes isoforms, allelic or splice variants, or post-translationally modified variants (e.g., glycosylation variants) of known FXI polypeptides, for instance of a FXI polypeptide having a sequence as depicted in SEQ ID NO: 7. It will be readily understood that the binding molecules of this disclosure may exhibit a binding affinity towards FXI variants comprising an amino acid sequence corresponding to amino acids 91 to 175 of SEQ ID NO: 7. In accordance with the foregoing, it is envisaged that the binding molecule is also capable of binding to and forming variable complexes with FXIa molecules and variants thereof, provided that they comprise the aforementioned amino acid stretch or amino acid positions corresponding thereto.

The binding molecules of this disclosure may also be capable of binding to FXI from numerous other mammalian species with or without preference to any species. These non-human FXI polypeptides are preferably encoded by a FXI gene or ortholog or paralog thereof and exhibit the same biological function as human FXI, even if they do not present as homodimers. Potential non-human primate protein targets of the binding molecules of this disclosure include polypeptides with UniprotAcc No. H2QQJ4 (Pan troglodytes, entry version 26 of 11 Nov. 2015), Uniprot Acc. No. H2PEX7 (Pongoabelii, entry version 27 of 11 Nov. 2015), Uniprot Ace. No. A0A0D9S2M6 (Chlorocebussabaeus, entry version 6 of 11 Nov. 2015), UniProt Acc. No. G3R2X1 (Gorilla gorilla gorilla, entry version 27 of 14 Oct. 2015), Uniprot Acc. No. 20 A0A096NC95 (Papio anubis, entry version 11 of 11 Nov. 2015), Uniprot Acc. No. G1RLE8 (Nomascusleucogenys, entry version 28 of 11 Nov. 2015), Uniprot Acc. No. G7PKF5 (Macaca fascicularis, entry version 13 of 14 Oct. 2015), UniProt Acc. No. G7MSF8 (Macaca mulatta, entry version 12 of 14 Oct. 2015). Other species include a range of mammalian FXI variants that possess the same conserved antigenic regions in the A2 domain as humans do. Variants of the aforementioned polypeptides are also envisaged as targets for the binding molecule of this disclosure. Envisaged non-human primate polypeptide targets recognized by the binding molecule of this disclosure are envisaged to comprise a sequence corresponding to amino acids 91 to 175 of SEQ ID NO: 7 or a sequence having at least 95%, 96%, 97%, 98%, or 99% sequence identity thereto. Thus, cross-species specific binding molecules directed against FXI, e.g., in non-human primates, are also provided herein. The term “cross-species recognition” or “interspecies specificity” as used herein thus means binding of a binding molecule described herein to the same target polypeptide in humans and non-human, e.g., non-human primate, species. 14E11 is a universal antibody meaning that it appears to form complexes with a wide variety and range of unrelated mammalian species. This aspect suggests that it binds to a highly conserved, or identical, sequence on the A2 domain of FXI. Because AB023 equivalency to 14E11 has been shown, the universality of AB023 allows for development of therapeutic antibodies with few species restrictions.

As set out herein, it is envisaged that the binding molecules described herein are also capable of binding to human or non-human mammalian FXIa. Thus, what is disclosed in the context of the binding molecule's binding characteristics as to FXI is preferably equally applicable to its binding characteristics as to FXIa, mutatis mutandis.

### Antibody

The binding molecule of this disclosure is envisaged to be an antibody. As is well known in the art, an antibody is an immunoglobulin molecule capable of specific binding to a target epitope through at least one epitope recognition site, located in the variable region of the immunoglobulin molecule. The terms “antibody”, “antibody molecule” and “immunoglobulin” are used interchangeably and in their broadest sense herein and may include native antibodies, monoclonal antibodies, polyclonal antibodies, multispecific antibodies (e.g., bispecific antibodies), (naturally occurring or synthetic) antibody derivatives, fragments, or variants, fusion proteins comprising an antigen-binding fragment of the required specificity and any other modified configuration of the antibody that comprises an antigen-binding site of the required specificity. Antibodies according to this disclosure are envisaged to be capable of binding to mammalian FXI as described herein, and preferably exhibit the advantageous characteristics of the antibody AB023 as set out herein.

### Native Antibody

A “native antibody” is a tetrameric glycoprotein. In a naturally-occurring native antibody, each tetramer is composed of two identical pairs of polypeptide chains, each pair having one “light” chain (about 25 kDa) and one “heavy” chain (about 50-70 kDa). The amino-terminal portion of each chain includes a “(hyper)variable” region of about 100 to 110 or more amino acids primarily responsible for antigen recognition. The hypervariable region comprises amino acid residues from a “complementarity determining region” or CDRs or “CDR regions”. “Framework” or FR residues are those variable domain residues other than the hypervariable region residues.

Both the light and heavy chains are divided into regions of structural and functional homology referred to as the “constant region” and the “variable region,” The terms “constant” and “variable” are used functionally. In this regard, it will be appreciated that the variable regions of both the light (V_{L}) and heavy (V_{H}) chains determine antigen recognition and specificity. The terms “V_{L}”, “V_{L} region”, and “V_{L} domain” are used interchangeably throughout the specification to refer to the variable region of the light chain. Similarly, the terms “V_{H}”, “V_{H} region” and “V_{H} domain” are used interchangeably herein to refer to the variable region of the heavy chain.

The terms “C_{L}”, C_{L} region” and “C_{L} domain” are used interchangeably herein to refer to the constant region of the light chain. The terms “C_{H}”, C_{H} region” and “C_{H} domain” are used interchangeably herein to refer to the constant region of the heavy chain and comprise the “C_{H1}”, C_{H2}”, and “C_{H3}” regions or domains. Conversely, the constant domains of the light chain (C_{L}) and the heavy chain (C_{H1}, C_{H2}, or C_{H3}) confer biological properties such as secretion, transplacental mobility, Fc receptor binding, complement binding, and the like. By convention the numbering of the constant region domains increases as they become more distal from the antigen binding site or amino-terminus of the antibody. The N-terminal portion is a variable region and at the C-terminal portion is a constant region; the C_{H3} and C_{L} regions actually comprise the carboxy-terminus of the heavy and light chain, respectively.

The variable region allows the antibody to selectively recognize and specifically bind epitopes on antigens. That is, the V_{L} and V_{H} region, or the subset of the complementarity determining regions (CDRs) within these variable domains, of an antibody combine to form the variable region that defines a three dimensional antigen binding site. This quaternary antibody structure forms the antigen binding site present at the end of each arm of the Y. More specifically, the antigen binding site is defined by three CDRs (CDR1, CDR2, CDR3, determined following Kabat numbering system) on each of the V_{H} and V_{L} regions. The three CDRs of the light chain are also designated CDR1 LC or CDR_{L1}, CDR2 LC or CDR_{L2} and CDR3 LC or CDR_{L3} herein. The three CDRs of the heavy chain are termed CDR1 HC or CDR_{H1}, CDR2 HC or CDR_{H2} and CDR3 HC or CDR_{H3}. In native antibodies, the six “complementarity determining regions” or “CDRs” or “CDR regions” present in each antigen binding domain are typically short, non-contiguous sequences of amino acids that are specifically positioned to form the antigen binding domain as the antibody assumes its three dimensional configuration in an aqueous environment.

Binding molecules and, e.g., antibodies, of this disclosure are envisaged to comprise a CDR1 of the light chain comprising the sequence KASQDVSTAVA (SEQ ID NO: 1); a CDR2 of the light chain comprising the sequence LTSYRNT (SEQ ID NO: 2); a CDR3 of the light chain comprising the sequence QQHYKTPYS (SEQ ID NO: 3); a CDR1 of the heavy chain comprising the sequence GYGIY (SEQ ID NO: 4); a CDR2 of the heavy chain comprising the sequence MIWGDGRTDYNSALKS (SEQ ID NO: 5); a CDR3 of the heavy chain comprising the sequence DYYGSKDY (SEQ ID NO: 6); and optionally a S241P modification. The skilled person will readily understand that the CDRs are located in the variable region of the light and heavy chain, respectively. A monoclonal antibody comprising the aforementioned CDRs has been evaluated as disclosed herein and designated “AB023” herein.

Binding molecules and preferred monoclonal antibodies, antigen-binding fragments thereof, variants thereof, and derivatives thereof, of this disclosure are envisaged to comprise a V_{L} region as depicted in SEQ ID NO: 8, and/or a V_{H} region as depicted in SEQ ID NO: 9. However, other combinations of V_{L} and V_{H} regions are also conceivable. Binding molecules and preferred monoclonal antibodies, antigen-binding fragments thereof, variants thereof, and derivatives thereof, of this disclosure are envisaged to comprise a light chain as depicted in SEQ ID NO: 10 or SEQ ID NO: 12, and/or a heavy chain as depicted in SEQ ID NO: 11 or SEQ ID NO: 13, However, other combinations of light and heavy chains are also conceivable.

The carboxy-terminal portion of each light and heavy chain defines a constant region primarily responsible for effector function. Immunoglobulins can be assigned to different classes depending on the amino acid sequence of the constant domain of their heavy chains. Heavy chains are classified as mu (μ), delta (Δ), gamma (γ), alpha (α), and epsilon (ε), and define the antibody's isotype as IgM, IgD, IgG, IgA, and IgE, respectively. Several of these may be further divided into subclasses or isotypes, e.g. IgG1, IgG2, IgG3, IgG4, IgA1 and IgA2. Different isotypes have different effector functions; for example, IgG1 and IgG3 isotypes often have antibody-dependent cellular cytotoxicity (ADCC) activity. Light chains are classified as either kappa or lambda (κ, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages. All immunoglobulin types, classes, and subclasses are within the scope of this disclosure. Antibodies according to this disclosure may be IgG antibodies, and specifically, IgG4 monoclonal antibodies.

### Monoclonal Antibodies

In accordance with this disclosure, envisaged are monoclonal antibodies, and antigen-binding fragments, variants, and derivatives thereof. The term “monoclonal antibody” as used herein refers to an antibody obtained from a population of substantially homogeneous antibodies, i.e., the individual antibodies comprising the population are identical except for possible naturally occurring mutations that may be present in minor amounts. In contrast to conventional (polyclonal) antibody preparations that may include different antibodies directed against different epitopes, monoclonal antibodies contain substantially similar epitope binding sites and may therefore be directed against the same epitope on an antigen. The term “monoclonal antibody” thus includes recombinant, chimeric, humanized, human, or Human Engineered™ monoclonal antibodies.

Various production methods for generating monoclonal antibodies are known in the art and are described, e.g., in Goding, Monoclonal Antibodies: Principles and Practice, pp. 116-227 (Academic Press, 1996). Suitable techniques include the hybridoma method first described by Kohler et al., Nature, 256: 495 (1975), recombinant DNA methods that involve isolation and sequencing of DNA encoding the monoclonal antibodies, and its subsequent introduction and expression in suitable host cells, and the isolation of antibodies from antibody phage libraries generated using the techniques first described in McCafferty et al., Nature, 348: 552-554 (1990).

### Chimeric Antibody

As set forth herein, the term “antibody” also includes chimeric antibodies. The phrase “chimeric antibody,” as used herein, refers to an antibody containing sequence derived from two different antibodies which may originate from different species. Specifically, the term refers to an antibody in which a portion or the heavy and/or light chain is identical with or homologous to corresponding sequences in antibodies derived from a species or belonging to an antibody class or subclass, while the remainder of the chain(s) is identical with or homologous to corresponding sequences in antibodies derived from another species or belonging to another antibody class or subclass, as well as fragments of such antibodies.

To put it differently, the term “chimeric antibody” will be held to mean any antibody wherein the antigen binding site is obtained or derived from a first species and the constant region (which may be intact, partial or modified in accordance with the instant invention) is obtained from a second species. For example, the antigen binding site may be from a non-human source (e.g., mouse or primate) and the constant region may be human. Chimeric antibodies may, for instance, comprise human and murine antibody fragments, e.g., human constant and mouse variable regions.

### Humanized Antibody

As set out herein, this disclosure, relates to a (monoclonal) humanized antibody, and antigen-binding fragments, variants, and derivatives thereof, derived from the mouse anti-FXI 14E11 (as performed by Abzena (f.k.a., Antitope Limited, Cambridge, GB) using both methods well known and used in the art as well as proprietary methodology).

A “humanized antibody” is generally defined as one that is (I) derived from a non-human source (e.g., a transgenic mouse which bears a heterologous immune system), which antibody is based on a human germline sequence; or (II) CDR-grafted, wherein the CDRs of the variable region are from a non-human origin, while one or more framework regions and/or part of the CDR sequence of the variable region are of human origin and, e.g., the constant region (if any) is of human origin.

The term “humanized antibody” thus includes antibodies in which the variable region in either the heavy chain, light chain, or both, of a human antibody is altered by at least partial replacement of one or more CDRs from a non-human antibody of known specificity, and optionally, by partial framework region replacement and sequence changing. In other words, an antibody in which one or more “donor” CDRs from a non-human antibody (such as mouse, rat, rabbit or non-human primate antibody) of known specificity is grafted into a human heavy or light chain framework region is referred to herein as a “humanized antibody.” It may not be useful to replace all of the CDRs with the complete CDRs from the donor variable domain to transfer the antigen binding capacity of one variable domain to another. Rather, only those residues useful to maintain the activity of the target binding site may be transferred.

In this disclosure, the precursor mouse 14E11 antibody was humanized by determining the 14E11 CDR residues and selecting from a database a human germline sequence with the best overall homology to the murine V_{H} and V_{L} sequences as an accepter human germline framework for grafting V_{H} and V_{L} CDRs, respectively, as detailed herein. Briefly, structural models of the chimeric anti-FXI antibody V regions were produced using Swiss PDB and analyzed in order to identify amino acids in the V region frameworks that may support the binding properties of the antibody. These amino acids were noted for incorporation into one or more variant CDR-grafted antibodies. Both the VH and Vκ sequences of the binding molecule contain typical framework residues and the CDR 1, 2 and 3 motifs are comparable to many murine antibodies.

The heavy and light chain V region amino acid sequences were compared against a database of human germline V region sequences in order to identify the heavy and light chain human sequences with the greatest degree of homology for use as human V region frameworks. A series of humanized heavy and light chain V regions were then designed by grafting the CDRs onto the frameworks and, as needed, by back mutation to the specific murine sequence of residues identified previously which may restore the antibody binding efficiency. Variant sequences with the lowest incidence of potential T cell epitopes were then selected as determined by application of Abzena's proprietary ex vivo technologies, EpiScreen™ (Jones T D, Hanlon M, Smith B J, Heise C T, Nayee P D, Sanders D A, Hamilton A, Sweet C, Unitt E, Alexander G, Lo K M, Gillies S D, Carr and Baker M P. The development of a modified human IFN-alpha2b linked to the Fc portion of human IgG1 as a novel potential therapeutic for the treatment of hepatitis C virus infection. J Interferon Cytokine Res. 2004 24(9):560-72; Jones T D, Phillips W J, Smith B J, Bamford C A, Nayee P D, Baglin T P, Gaston J S and Baker M P. Identification and removal of a promiscuous CD4+ T cell epitope from the CI domain of factor VIII. J Thromb Haemost. 2005 3(5):991-1000). For the purposes of this disclosure, humanized antibodies that have been CDR optimized (“germlined”) are comprised within the term “humanized” antibodies.

The framework regions (FR) within the variable region in a heavy chain, light chain, or both, of a humanized antibody may be comprised of substantially all or all residues of human origin, in which case these framework regions of the humanized antibody are referred to as “fully human framework regions.” A human framework region that comprises a mixture of human and donor framework residues, and is referred to herein as a “partially human framework region.” Furthermore, humanized antibodies may comprise residues that are neither found in the recipient antibody nor in the donor antibody. These modifications are made to further refine antibody performance (e.g., to obtain desired affinity).

In general, the humanized antibody will thus comprise substantially all of at least one, and in some cases two, variable regions, in which all or part of the CDRs correspond to those of a non-human immunoglobulin and all or substantially all of the las are those of a human immunoglobulin sequence. The humanized antibody optionally also will comprise at least a portion of an immunoglobulin constant region (Fc), e.g., that of a human immunoglobulin.

### Human Antibody

A “human” antibody is hereby defined as one that is not chimeric or “humanized” and not from (either in whole or in part) a non-human species. A human antibody or functional antibody fragment can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising an amino acid sequence utilizing the data obtained therefrom. Another example of a human antibody or functional antibody fragment is one that is encoded by a nucleic acid isolated from a library of antibody sequences of human origin (i.e., such library being based on antibodies taken from a human natural source).

### Fragments, Variants and Derivatives

As set out herein, this disclosure encompasses full-length antibodies as well as antigen-binding fragments, variants, and derivatives thereof

### Fragments

The term “antibody fragment” refers to a polypeptide derived from a “parent” antibody and retaining its basic structure and function. An antibody fragment is hence preferably capable of binding to its specific antigen, i.e., FXI. Furthermore, an antibody fragment according to this disclosure comprises the minimum structural requirements of an antibody which allow for antigen binding. This minimum requirement may be, e.g., defined by the presence of at least the three light chain CDRs (i.e., CDR1, CDR2 and CDR3 of the V_{L} region, i.e., CDR_{L1}, CDR_{L2} and CDR_{L3}) and/or the three heavy chain CDRs (i.e., CDR1, CDR2 and CDR3 of the Vₖ region, i.e., CDR_{H1}, CDR_{H2} and CDR_{H3}). Thus, the term “antibody fragment” refers to a “functional” or “antigen-binding” polypeptide that retains the antigen-binding site (i.e., the CDRs and optionally (part of) the FR) of a “parent” antibody. Antibody fragments of this disclosure may be derived from, e.g., monoclonal, recombinant, chimeric, humanized, and human “parent” antibodies.

Preferred antigen binding antibody fragments comprise at least one of, preferably all of, a CDR1 of the light chain comprising the sequence KASQDVSTAVA (SEQ. ID NO: 1): a CDR2 of the light chain comprising the sequence LTSYRNT (SEQ ID NO: 2); a CDR3 of the light chain comprising the sequence QQHYKTPYS (SEQ ID NO: 3); a CDR1 of the heavy chain comprising the sequence GYGIY (SEQ ID NO: 4); a CDR2 of the heavy chain comprising the sequence MIWGDGRTDYNSALKS (SEQ ID NO: 5); a CDR3 of the heavy chain comprising the sequence DYYGSKDY (SEQ ID NO: 6).

In accordance with the foregoing, the term “antigen binding antibody fragments” may refer to fragments of, e.g., full-length antibodies, such as, (s)dAb, F, Fd, Fab, Fab′, F(ab′)2 or “r IgG” (“half antibody”). Antibody fragments according to this disclosure may also be modified fragments of antibodies such as scFv, di-scFv or bi(s)-scFv, scFv-Fc, scFv-zipper, scFab, Fab2, Fab3, diabodies, single chain diabodies, tandem diabodies (Tandab's), tandem di-scFv, tandem tri-scFv, “minibodies” exemplified by a structure which is as follows: (V_{H}-V_{L}-CH₃)₂, (scFv-CH₃)₂ or (scFv-CH₃-scFv)₂, multibodies such as triabodies or tetrabodies. Furthermore, the definition of the term “antibody fragments” includes constructs comprising the fragments, i.e., monovalent, bivalent and polyvalent/multivalent constructs, and thus, monospecific constructs, specifically binding to only one target antigen, as well as bispecific and polyspecific/multispecific constructs which specifically bind more than one target antigens, e.g., two, three, or more, through distinct antigen binding sites. Moreover, the definition of the term “antibody fragments” includes molecules consisting of only one polypeptide chain as well as molecules consisting of more than one polypeptide chain, which chains can be either identical (homodimers, homotrimers or homo oligomers) or different (heterodimer, heterotrimer or heterooligomer).

Antibody fragments may be produced by recombinant DNA techniques or by enzymatic or chemical cleavage of intact antibodies. Methods for producing such fragments are well-known in the art.

### Variants

The term “variant” refers to polypeptides comprising the amino acid sequence of a “parent” binding molecule, such as an antibody or antibody fragment, but containing at least one amino acid modification (e.g., a substitution, deletion, or insertion) as compared to the “parent” amino acid sequence, provided that the variants are still capable of (specifically) binding to FXI, preferably the A2 domain of human FXI as depicted in SEQ ID NO: 7, and preferably exhibits similar or even improved characteristics as compared to the antibody AB023. Variants of the binding molecules of this disclosure, e.g., antibodies and antibody fragments, may be prepared by introducing appropriate nucleotide changes into the nucleic acids encoding the antibody or antibody fragment, or by peptide synthesis. Generally, the aforementioned amino acid modifications may be introduced into, or present in, the variable region or the constant region, under the premise that two or more CDRs of the variants cumulatively comprise 10, 11, 12, 13 or 14 amino acid substitutions as compared to the AB023 CDRs as depicted in SEQ. ID NOS: 1, 2, 3, 4, 5, and 6. Amino acid modifications can for example be introduced in order to modulate antibody properties like thermodynamic stability, solubility or viscosity which affect pharmaceutical development (“sequence optimization”).

As set out herein, amino acid modifications include, for example, deletions from, and/or insertions into, and/or substitutions of residues within the amino acid sequences of binding molecules described herein, preferably the antibodies or antigen binding antibody fragments. Any combination of deletion, insertion, and substitution can be introduced into the “parent” amino acid sequence in order to arrive at the final product, provided that it possesses the desired characteristics as set out herein. The amino acid modifications also may alter post-translational processes of the binding molecules, such as changing the number or position of glycosylation sites.

For example, variants may comprise 1, 2, 3, 4, 5, or 6 amino acids be inserted or deleted in each of the CDRs (of course, dependent on their length), while 1, 2, 3, 4, 5, 6, 7, 8, 9, 10, 11, 12, 13, 14, 15, 16, 17, 18, 19, 20, or 25 amino acids may be inserted or deleted in each of the FRs. Amino acid sequence insertions envisaged herein include, e.g., amino- and/or carboxyl-terminal fusions ranging in length from 1, 2, 3, 4, 5, 6, 7, 8, 9 or 10 residues to polypeptides containing a hundred or more residues, as well as intra-sequence insertions of single or multiple amino acid residues. An insertional variant of a binding molecule, e.g., an antibody or antibody fragment, of this disclosure may include a fusion product of an antibody or antibody fragment and an enzyme or another functional polypeptide (e.g., which may increase the serum half-life of the binding molecule, e.g., antibody or antibody fragment).

Amino acid substitutions can be introduced into the CDRs of the heavy and/or light chain, e.g., the hypervariable regions, or the FR regions in the heavy and/or light chain. Envisaged herein are conservative amino acid substitutions that may be made, for instance, on the basis of similarity in polarity, charge, solubility, hydrophobicity, hydrophilicity, and/or the amphipathic nature of the residues involved.

Alternative variants may comprise, 1, 2, 3, 4, 5, 6, 7, 8, 9, or 10, amino acids substituted in the CDRs as compared to the CDRs as depicted in SEQ ID NO: 1-6, while 1, 2, 3, 4, 5, 6, 7, 8, 9, 10, 11, 12, 13, 14, 15, 16, 17, 18, 19, 20, or 25 amino acids may be substituted in the framework regions (FRs), depending on the length of the CDR or FR.

Generally, if amino acids are substituted in one or more or all of the CDRs of the heavy and/or light chain, it is preferred that the then-obtained “variant” sequence is at least 80%, still more preferably at least 90% and most preferably at least 95%, 96%, 97%, 98% or 99% identical to the “parent” CDR sequence. The length of the CDR thus influences the number of possible amino acid substitutions so that the variant sequence is still encompassed by this disclosure. For example, a CDR having 5 amino acids is preferably 80% identical to its substituted sequence in order to have at least one amino acid substituted. Accordingly, the CDRs of the antibody construct may have different degrees of identity to their substituted sequences, e.g., CDR_{L1} may have 80%, while CDR_{L3} may have 90%.

Preferred substitutions (or replacements) are conservative substitutions. However, any substitution (including non-conservative substitution or one or more from the exemplary substitutions) is envisaged as long as the antibody construct retains its capability to bind FXI and/or its CDRs have an identity to the then substituted sequence of at least 80%, still more preferably at least 90% and most preferably at least 95%, 96%, 97%, 98% or 99%.

As used herein, the term “sequence identity” indicates the extent to which two (nucleotide or amino acid) sequences have identical residues at the same positions in an alignment, and is often expressed as a percentage. Preferably, identity is determined over the entire length of the sequences being compared. Thus, two copies of exactly the same sequence have 100% identity, but sequences that are less highly conserved, and have deletions, additions, or replacements, may have a lower degree of identity. Those skilled in the art will recognize that several algorithms are available for determining sequence identity using standard parameters, for example, Blast (Altschul, et al. (1997) Nucleic Acids Res. 25:3389-3402), Blast2 (Altschul, et al. (1990) J. Mol. Biol. 215:403-410), Smith-Waterman (Smith, et al. (1981) J. Mol. Biol. 147:195-197), and Clustal W.

The term “sequence homology” indicates the similarity of two (nucleotide or amino acid) sequences attributed to descent from a common ancestor. Homologous biological components (genes, proteins, structures) are called homologs and include orthologs and paralogs.

Preferred binding molecule variants of this disclosure have a sequence identity or homology in the CDR regions of at least 80%, still more preferably at least 90% and most preferably at least 95%, 96%, 97%, 98%, 99% or almost 100% and exhibit a comparable or improved binding affinity to FXI and/or a comparable or improved biological activity as compared to binding molecules comprising the “parent” CDRs, preferably, SEQ ID NO: 1, 2, 3, 4, 5, and 6.

Moreover, the nucleic acid sequence homology or similarity between the nucleotide sequences encoding individual variant CDRs and the nucleotide sequences depicted herein are at least 80%, and preferably with increasing homologies or identities of at least 81%, 82%, 83%, 84%, 85%, 86%, 87%, 88%, 89%, 90%, 91%, 92%, 93%, 94%, 95%, 96%, 97%, 98%, or 99%, and almost 100%.

Besides in the CDRs and FRs, amino acid modifications may also be introduced into the Fc part of a binding molecule, which is preferably a monoclonal antibody or antigen-binding fragment thereof. Such modifications can be used in order to modulate functional properties or the antibody, e.g., interactions with the complement proteins such as C1q and/or Fc receptors on other immune cells, or to modulate serum half-life or antigen-dependent cellular cytotoxicity (ADCC). Thus, mutations for modification of effector functions may be introduced into the Fc domains using routine methods known in the art. Exemplary modifications include Asn297→Ala297 and Asn297→Gln297 resulting in a glycosylation of IgG1, or Lys322→Ala322 and optionally Leu234→Ala234 and Leu235→Ala234 which have been reported to reduce or abolish antibody-derived cell-mediated cytotoxicity (ADCC) and/or complement-derived cytotoxicity (CDC).

### Derivatives

The term “binding molecule” also encompasses derivatives. Envisaged herein are derivatives of antibodies or antibody fragments as disclosed elsewhere herein. The term “derivative” generally refers to a binding molecule that has been covalently modified to introduce an additional functionality. Covalent modifications of the binding molecules are generally, but not always, done post-translationally, and can be introduced into the binding molecule by reacting specific amino acid residues of the molecule with an organic derivatizing agent that is capable of reacting with selected side chains or the N- or C-terminal residues. Derivatization of binding molecules can be used to attach therapeutic or diagnostic agents, labels, groups extending the serum half-life of the molecule, or insertion of non-natural amino acids. Possible chemical modifications of the binding molecules of this disclosure include, for example, acylation or acetylation of the N-terminal end, or amidation or esterification of the C-terminal end or, alternatively, on both. Chemical modifications such as alkylation (e.g., methylation, propylation, butylation), arylation, and etherification are also envisaged.

### Serum Half-Life Extension

Examples for means to extend serum half-life of the binding molecules, and preferably antibodies and antigen-binding fragments thereof of this disclosure, include the attachment of peptides or protein domains binding to other proteins in the human body (such as serum albumin, the immunoglobulin Fc region or the neonatal Fc receptor (FcRn). Further conceivable modifications to extend the serum half-life comprise the extension of an amino group with polypeptide chains of varying length (e.g., XTEN technology or PASylation®), the conjugation of non-proteinaceous polymers, including, but not limited to, various polyols such as polyethylene glycol (PEGylation), polypropylene glycol, polyoxyalkylenes, or copolymers of polyethylene glycol and polypropylene glycol, or of carbohydrates, such as hydroxyethyl starch (e.g., HESylation®) or polysialic acid (e.g., PolyXen® technology). In addition, as is known in the art, amino acid substitutions may be made in various positions within the binding molecule in order to facilitate the addition of the polymers.

### Glycosylation

Another type of covalent modification of the binding molecules, and preferably antibodies and antigen-binding fragments thereof of this disclosure, comprises altering its glycosylation pattern. As is known in the art, glycosylation patterns can depend on both the amino acid sequence of the molecule (e.g., the presence or absence of glycosylation amino acid residues), or the host cell or organism in which the protein is produced. Glycosylation of polypeptides may be either N-linked or O-linked. N-linked refers to the attachment of the carbohydrate moiety to the side chain of an asparagine residue. Addition of N-linked glycosylation sites to the binding molecule is conveniently accomplished by altering the amino acid sequence such that it contains one or more tri-peptide sequences selected from asparagine-X-serine and asparagine-X-threonine (where X is any amino acid except proline). O-linked glycosylation sites may be introduced by the addition of, or substitution by, one or more serine or threonine residues to the starting sequence.

Another means of glycosylation of the binding molecule is by chemical or enzymatic coupling of glycosides to the protein. These procedures are advantageous in that they do not require production of the protein in a host cell that has glycosylation capabilities for N- and O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, or hydroxyproline, (e) aromatic residues such as those of phenylalanine, tyrosine, or tryptophan, or (f) the amide group of glutamine.

Similarly, deglycosylation (i.e., removal of carbohydrate moieties present on the binding molecule) may be accomplished chemically, e.g., by exposure of the binding molecule to trifluoromethanesulfonic acid, or enzymatically by employing endo- and exo-glycosidases.

### Labeling

Further potential covalent modifications of the binding molecules of this disclosure comprise the addition of one or more labels. The labeling group may be coupled to the binding molecule via spacers of various lengths to reduce potential steric hindrance. Various methods for labeling proteins are known in the art and can be used in performing this disclosure. The term “label” or “labeling group” refers to any detectable label. In general, labels fall into a variety of classes, depending on the assay in which they are to be detected. The following exemplary labels include, but are not limited to: isotopic labels, which may be radioactive or heavy isotopes, such as radioisotopes or radionuclides (e.g., 3H, 14C, 15N, 35S, 89Zr, 90Y, 99Tc, 111In, 125I, 131I); magnetic labels (e.g., magnetic particles); redox active moieties; optical dyes (including, but not limited to, chromophores, phosphors and fluorophores) such as fluorescent groups (e.g., FITC, rhodamine, lanthanide phosphors), chemiluminescent groups, and fluorophores which can be either “small molecule” fluorophores or proteinaceous fluorophores; enzymatic groups (e.g. horseradish peroxidase, β-galactosidase, luciferase, alkaline phosphatase; biotinylated groups; or predetermined polypeptide epitopes recognized by a secondary reporter (e.g., leucine zipper pair sequences, binding sites for secondary antibodies, metal binding domains, epitope tags, etc.).

### ADCs

It is also conceivable to add a drug, such as a small molecule compound, to the binding molecules, and preferably antibodies or antigen-binding fragments thereof, of this disclosure. Antibody Drug Conjugates” (“ADC”) are antibodies or antigen-binding fragments thereof linked to a drug or agent. The linkage can be established through covalent bonds, or non-covalent interactions such as through electrostatic forces. Various linkers, known in the art, can be employed in order to form the ADC as is known in the art.

### Affinity Tags

The binding molecule, and preferably antibody or antigen-binding fragments thereof of this disclosure, may also comprise additional domains, which may aid in purification and isolation of the molecule (affinity tags). Non-limiting examples of such additional domains comprise peptide motives known as Myc-tag, HAT-tag, HA-tag, TAP-tag, GST-tag, chitin binding domain (CBD-tag), maltose binding protein (MBP-tag), Flag-tag, Strep-tag and variants thereof (e.g., StrepII-tag), and His-tag.

The aforementioned fragments, variants and derivatives may be further adapted in order to improve, e.g., their antigen binding properties. For instance, F(ab′)₂ or Fab may be engineered to minimize or completely remove the intermolecular disulphide interactions that occur between the C_{H1} and C_{L} domains. Fv polypeptides may further comprise a polypeptide linker between the V_{H} and V_{L} domains that enables the Fv to form the desired structure for antigen binding. The Fab fragment also contains the constant domain of the light chain and the first constant region (CH₁) of the heavy chain. Fab fragments differ from Fab′ fragments by the addition of a few residues at the carboxy terminus of the heavy chain CH₁ region including one or more cysteines from the antibody hinge region. Fab′-SH is the designation herein for Fab′ in which the cysteine residue(s) of the constant region bear a free thiol group. F(ab′)2 antibody fragments originally were produced as pairs of Fab′ fragments which have hinge cysteine residues between them.

The binding molecules of this disclosure may be provided in “isolated” or “substantially pure” form. “Isolated” or “substantially pure” when used herein means that the binding molecule has been identified, separated and/or recovered from a component of its production environment, such that the “isolated” binding molecule is free or substantially free of other contaminant components from its production environment that might interfere with its therapeutic or diagnostic use. Contaminant components may include enzymes, hormones, and other proteinaceous or non-proteinaceous solutes. “Isolated” binding molecules will thus be prepared by at least one purification step removing or substantially removing these contaminant components. The aforementioned definition is equally applicable to “isolated” polynucleotides, mutatis mutandis.

### Specific Binding

The binding molecules of this disclosure, e.g., antibodies and antigen-binding fragments thereof, are advantageously capable of binding to various mammalian FXI, preferably human FXI, comprising or consisting of an amino acid sequence as depicted in SEQ ID NO: 7. The terms “binding to” and “recognizing” in all grammatical forms are used interchangeably herein. Preferably, the binding molecules specifically bind to FXI. The term “specifically binds” generally indicates that a binding molecule, e.g., an antibody or antigen-binding fragment thereof as described herein, binds via its antigen binding site more readily to its intended target epitope than to a random, unrelated non-target epitope. The term “specifically binds” indicates that the affinity of the binding molecule will be at least about 5 fold, preferably 10 fold, more preferably 25-fold, even more preferably 50-fold, and most preferably 100-fold or more, greater for its target epitope than its affinity for a non-target epitope.

Thus, a binding molecule, i.e., an antibody, or antigen-binding fragment, variant, or derivative thereof, may be considered to specifically bind to its target epitope if it binds the epitope with a dissociation constant (K_{D}) that is less than the antibody's K_{D} for a non-target epitope. Binding molecules of this disclosure may also be described in terms of their binding affinity to mammalian FXI, preferably human FXI. The term “affinity” or “binding affinity” refers to the strength of the binding of an individual epitope with an antigen-binding domain (i.e., the CDRs of the binding molecule). The affinity of the binding of a given binding molecule to its specific epitope is often determined by measurement of the equilibrium association constant (ka) and equilibrium dissociation constant (kd) and calculating the quotient of kd to ka (K_{D}=kd/ka). Binding affinities may be readily determined using conventional techniques, such as by equilibrium dialysis; by using the BIAcore 2000 instrument; by radioimmunoassay using radiolabeled target antigen; or by another method known to the skilled artisan. The affinity data may be analyzed, for example, by the method described in Kaufman R J and Sharp P A. (1982) J Mol Biol. 159:601-621. Preferred binding affinities of the inventive binding molecules include those with a dissociation constant or K_{D} less than 5×10⁻⁶ M, 10⁻⁶ M, 5×10⁻⁷ M, 10⁻⁷ M, 5×10⁻⁸ M, 10⁻⁸ M, 5×10⁻⁹ M, 10⁻⁹ M, 5×10⁻¹⁰ M, 10⁻¹⁰ M, 5×10⁻¹¹ M, 10⁻¹¹ M, 5×10⁻¹² M, 10⁻¹² M, 5×10⁻¹³ M, 10⁻¹³ M, 5×10⁻¹⁴ M, 10⁻¹⁴ M, 5×10⁻¹⁵M, or 10⁻¹⁵M.

### Cross-Reactivity

The term “specifically binds” however does not exclude that the binding molecules (specifically) binding to human FXI cross-reacts with a FXI protein from a different species. Accordingly, binding molecules of this disclosure are also capable of binding to FXI from other mammalian species.

“Cross-species” binding or recognition means binding of a binding domain described herein to the same target antigen in humans and non-human species. Thus, “cross-species specificity” is to be understood as an interspecies reactivity to FXI expressed in different species, but not to an antigen other than FXI. For example, a binding domain which binds to human FXI, preferably to the A2 domain comprising amino acids 91 to 175 of the amino acid sequence shown in SEQ ID NO:7, also binds to other non-human FXI, and preferably to a region characteristic of, corresponding or similar to amino acids 91 to 175 of the amino acid sequence shown in SEQ ID NO: 7.

### Biological Activity

The binding molecules provided herein are envisaged to be biologically active, i.e., to bind to mammalian FXI and/or FXIa and block some of its respective biological functions. Specifically, “biologically active” binding molecules according to this disclosure form an immune complex with FXI and the FXI-AB023 immune complex cannot be efficiently activated by FXIIa or autoactivated. However, the immune complex can still be activated by thrombin. Once the FYI-AB023 complex is activated to FXIa-AB023, its activity towards FXII to convert the FXII zymogen to FXIIa is reduced, yet, the activated complex can perform, without loss-of function, the conversion of FIX to Factor IXa, preferably resulting in a complete or partial inhibition of contact activation while preserving thrombin-mediated hemostatic feedback activation of blood. Binding of the biologically active binding molecules to their target FXI and/or FXIa is thus envisaged to result in an anticoagulatory activity, e.g., in an assay that initiates coagulation through the contact activation complex. To put it differently, it is envisaged that the binding molecules according to this disclosure exert their beneficial function via a) binding to FXI, thereby blocking its conversion into its active form FXIa by FXIIa or autoactivation, and/or b) binding to FXIa, thereby reducing its binding to and activating of FXII. Binding molecules of this disclosure thereby preferably interrupt the contact activation complex, and thereby reduce contact activation-associated pathological processes, including, for example, inflammation and thrombosis, advantageously without impairing those hemostatic processes that are independent of the contact activation complex.

The anticoagulant activity of a binding molecule can be determined in vitro as described herein. Briefly, the activated partial thromboplastin time (aPTT), of normal human or other mammalian plasma which measures contact system activation-dependent thrombin generation, is determined in the presence of varying concentrations of the binding molecule or the corresponding solvent using a commercial test kit (SynthASil reagent from Instrumentation Laboratories, Bedford, Mass.). The test compounds are incubated with the plasma that normally contains endogenous FXI in a concentration range of 20 to 45 nM and the SynthASil reagent (colloidal silica activator) at 37° C. for about 3 minutes. Coagulation is then started by addition of 25 mM calcium chloride, and the time when coagulation occurs is determined, and the concentration of the test substance which effects about a 2.0 fold prolongation of the aPTT is determined. It is envisaged that the binding molecules of this disclosure lead to a 1.5-fold, 2.0-fold, or higher, prolongation of the aPTT.

Advantageously, binding molecules, and preferably monoclonal antibodies and antigen binding fragments thereof, according to this disclosure exhibit the above-mentioned biological properties and are therefore promising new agents for inhibition of thrombosis and inflammation. Because the binding molecules are envisaged to specifically bind to FXI, it is envisaged that they do not, or do not severely, compromise hemostasis and thereby preferably do not increase the risk of bleeding.

### Polynucleotide

This disclosure further provides a polynucleotide/nucleic acid molecule encoding a binding molecule or a V_{H} or a V_{L} domain of this disclosure.

The term “polynucleotide” as used herein comprises polyribonucleotides and polydeoxyribonucleotides, e.g., modified or unmodified RNA or DNA, each in single-stranded and/or multi-stranded (e.g., double-stranded) form, linear or circular, or mixtures thereof, including hybrid molecules. A polynucleotide may comprise a conventional phosphodiester bond or a non-conventional bond (e.g., an amide bond, such as found in peptide nucleic acids (PNA)). The polynucleotides of this disclosure may also contain one or more modified bases, such as, for example, tritylated bases and unusual bases such as inosine. Other modifications, including chemical, enzymatic, or metabolic modifications, are also conceivable, as long as a binding molecule of this disclosure can be expressed from the polynucleotide. The polynucleotide may be provided in isolated form as defined herein. A polynucleotide may include regulatory sequences such as transcription control elements (including promoters, enhancers, operators, repressors, and transcription termination signals), ribosome binding site, introns, or the like.

The present invention provides a polynucleotide comprising, or consisting of a nucleic acid encoding an immunoglobulin heavy chain domain (V_{H} region), where at least one of the CDRs of the V_{H} region has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 8. A binding molecule comprising the encoded CDRs or V_{H} domains is envisaged to be capable of binding to FXI and by complex formation between FXI and the binding molecule preferably exhibit the desired biological activities as described herein.

This disclosure provides a polynucleotide comprising, or consisting of, a nucleic acid encoding an immunoglobulin light chain domain (V_{L} region), where at least one of the CDRs of the V_{L} region has an amino acid sequence that is at least about 80%, about 85%, about 90%, about 91%, about 92%, about 93%, about 94%, about 95%, about 96%, about 97%, about 98%, about 99%, or 100% identical to SEQ ID NO: 9. A binding molecule comprising the encoded CDRs or V_{L} regions is envisaged to be capable of binding to FXI and by complex formation between FXI and the binding molecule preferably exhibit the desired biological activities as described herein.

The polynucleotides described herein may or may not comprise additional nucleotide sequences, encoding, e.g., a signal peptide to direct secretion of the encoded polypeptide, antibody constant regions as described herein, or other heterologous polypeptides as described herein. Such polynucleotides may thus encode fusion polypeptides, fragments, variants, and other derivatives of the binding molecules described herein.

Also, this disclosure includes compositions comprising one or more of the polynucleotides described above. Also provided herein are compositions comprising a first polynucleotide and second polynucleotide wherein the first polynucleotide encodes a V_{H} region as described herein and wherein the second polynucleotide encodes a V_{L} region as described herein, specifically a composition which comprises, or consists of a V_{H} region depicted in SEQ ID NO: 8, and/or a V_{L} region depicted in SEQ ID NO:9.

### Production of Polynucleotides

Polynucleotides of this disclosure may be produced by routine methods known in the art. For example, if the nucleotide sequence of the binding molecule is known, a polynucleotide encoding the binding molecule may be assembled from chemically synthesized oligonucleotides, annealing and ligating of those oligonucleotides, and then amplification of the ligated oligonucleotides by PCR. A polynucleotide encoding a binding molecule may be obtained from a suitable source (e.g., a cDNA library, or a nucleic acid such as a poly(A)+ mRNA isolated from any tissue or cells expressing the binding molecule such as hybridoma cells) by PCR amplification using synthetic primers hybridizable to the 3′ and 5′ ends of the sequence or by cloning using an oligonucleotide probe specific for the gene sequence to identify, e.g., a cDNA clone from a cDNA library that encodes the binding molecule.

Once the nucleotide sequence and corresponding amino acid sequence of the binding molecule is determined, its nucleotide sequence may be modified using methods well known in the art for the manipulation of nucleotide sequences, e.g., recombinant DNA techniques, site directed mutagenesis, PCR, etc., thereby introducing one or more nucleotide substitutions, additions or deletions into the polynucleotide sequence (see, for example, the techniques described in J. Sambrook et al., Molecular Cloning: A Laboratory Manual (4th edition), Cold Spring Harbor Laboratory, Cold Spring Harbor Laboratory Press, New York (2012)) to generate non-naturally occurring fragments, variants or derivatives of the binding molecule, i.e., the monoclonal anti-FXI antibody described herein (e.g., an immunoglobulin heavy chain region or light chain region).

### Vectors

Further provided herein is a vector comprising the polynucleotide as described herein. The polynucleotide encodes a binding molecule of this disclosure, preferably a monoclonal antibody or antigen binding fragment thereof. A “vector” is a nucleic acid molecule used as a vehicle to transfer (foreign) genetic material into a host cell where it can for instance be replicated and/or expressed.

The terra “vector” encompasses, without limitation, plasmids, viral vectors (including retroviral vectors, lentiviral vectors, adenoviral vectors, vaccinia virus vectors, polyoma virus vectors, and adenovirus-associated vectors (AAV)), phages, phagemids, cosmids and artificial chromosomes (including BACs and YACs). The vector itself is generally a nucleotide sequence, commonly a DNA sequence that comprises an insert (transgene) and a larger sequence that serves as the “backbone” of the vector. Engineered vectors may comprise an origin for autonomous replication in the host cells (if stable expression of the polynucleotide is desired), selection markers, and restriction enzyme cleavage sites (e.g. a multiple cloning site, MCS). Vectors may additionally comprise promoters, genetic markers, reporter genes, targeting sequences, and/or protein purification tags. Vectors called expression vectors (expression constructs) are specifically designed for the expression of the transgene in the target cell, and generally have control sequences. Large numbers of suitable vectors are known to those of skill in the art and many are commercially available. Examples of suitable vectors are provided in J. Sambrook et al., Molecular Cloning: A Laboratory Manual (4th edition), Cold Spring Harbor Laboratory, Cold Spring Harbor Laboratory Press, New York (2012).

### Targeting Vectors

Targeting vectors can be used to integrate a polynucleotide into the host cell's chromosome (Sambrook et al., 2012). Briefly, suitable means include homologous recombination or use of a hybrid recombinase that specifically targets sequences at the integration sites. Targeting vectors may be circular and linearized before use for homologous recombination. As an alternative, the foreign polynucleotides may be DNA fragments joined by fusion PCR or synthetically constructed DNA fragments which are then recombined into the host cell. It is also possible to use heterologous recombination which results in random or non-targeted integration.

### Production

### Expression Vectors

“Expression vectors” or “expression constructs” can be used for the transcription of heterologous polynucleotide sequences, for instance those encoding the binding molecules of this disclosure, and translation of their mRNA in a suitable host cell. This process is also referred to herein as “expression” of the binding molecules of this disclosure. Besides an origin of replication, selection markers, and restriction enzyme cleavage sites, expression vectors may include one or more regulatory sequences operably linked to the heterologous polynucleotide to be expressed.

The term “regulatory sequence” refers to a nucleic acid sequence necessary for the expression of an operably linked coding sequence of a (heterologous) polynucleotide in a host organism, and thus, include transcriptional and translational regulatory sequences. Regulatory sequences required for expression of heterologous polynucleotide sequences in prokaryotes include, e.g., promoter(s), optionally operator sequence(s), and ribosome binding site(s). In eukaryotes, promoters, polyadenylation signals, enhancers and optionally splice signals may be required. Moreover, specific initiation and secretory signals also may be introduced into the vector in order to allow for secretion of the polypeptide of interest into the culture medium.

A nucleic acid is “operably linked” when it is placed into a functional relationship with another nucleic acid sequence, e.g., on the same polynucleotide molecule. For example, a promoter is operably linked with a coding sequence of a heterologous gene when it is capable of effecting the expression of that coding sequence. The promoter may be placed upstream of the gene encoding the polypeptide of interest and regulate the expression of the gene.

Exemplary regulatory sequences for mammalian host cell expression include viral elements that direct high levels of protein expression in mammalian cells, such as promoters and/or enhancers derived from cytomegalovirus (CMV) (such as the CMV promoter/enhancer), Simian Virus 40 (SV40) (such as the SV40 promoter/enhancer), adenovirus, (e.g., the adenovirus major late promoter (AdMLP)) and polyoma. For further description of viral regulatory elements, and sequences thereof, see for example: U.S. Pat. No. 5,168,062 to Stinski; U.S. Pat. No. 4,510,245 to Cousens et al.; and U.S. Pat. No. 4,968,615 to Koszinowski et al. Expression vectors may also include origins of replication and selectable markers.

Vectors of this disclosure may further comprise one or more selection markers. Suitable selection markers for use with eukaryotic host cells include, without limitation, the herpes simplex virus thymidine kinase (tk), hypoxanthine-guanine phosphoribosyltransferase (hgprt), and adenine phosphoribosyltransferase (aprt) genes. Other genes include dhfr (methotrexate resistance), gpt (mycophenolic acid resistance) neo (G-418 resistance) and hygro (hygromycinresistance). Vector amplification can be used to increase expression levels. In general, the selection marker gene can either be directly linked to the polynucleotide sequences to be expressed, or introduced into the same host cell by cotransformation.

In view of the above, this disclosure, thus, further provides one or more of the polynucleotide sequences described herein which may be inserted into a vector. This disclosure, thus, provides replicable vectors comprising a nucleotide sequence encoding a binding molecule of this disclosure, or a heavy or light chain thereof, or a heavy or light chain variable domain, operably linked to a promoter. Such vectors may include the nucleotide sequence encoding the constant region of the binding molecule and the variable domain of the binding molecule may be cloned into such a vector for expression of the entire heavy or light chain.

### Host Cell

In general, a variety of host cells may be employed to express the binding molecule of this disclosure from an expression vector. As used herein, a “host cell” refers to a cell which can be or has/have been recipients of polynucleotides or vectors or encoding the binding molecule of this disclosure, Specifically, a host cell may further be capable of expressing and optionally secreting the binding molecule. In descriptions of processes for obtaining binding molecules from host cells, the terms “cell” and “cell culture” are used interchangeably to denote the source of a binding molecule unless it is clearly specified otherwise. The term “host cell” also includes “host cell lines”.

In general, the term includes prokaryotic or eukaryotic cells, and also includes without limitation bacteria, yeast cells, fungi cells, plant cells, and animal cells such as insect cells and mammalian cells, e.g., murine, rat, macaque, or human cells. Polynucleotides and/or vectors of this disclosure can be introduced into the host cells using routine methods known in the art, e.g., by transfection, transformation, or the like.

“Transfection” is the process of deliberately introducing nucleic acid molecules or polynucleotides (including vectors) into target cells. The term is mostly used for non-viral methods in eukaryotic cells. Transduction is often used to describe virus-mediated transfer of nucleic acid molecules or polynucleotides. Transfection of animal cells may involve opening transient pores or “holes” in the cell membrane, to allow the uptake of material. Transfection can be carried out using calcium phosphate, by electroporation, by cell squeezing or by mixing a cationic lipid with the material to produce liposomes, which fuse with the cell membrane and deposit their cargo inside. Exemplary techniques for transfecting eukaryotic host cells include lipid vesicle mediated uptake, heat shock mediated uptake, calcium phosphate mediated transfection (calcium phosphate/DNA co-precipitation), microinjection, and electroporation.

The term “transformation” is used to describe non-viral transfer of nucleic acid molecules or polynucleotides (including vectors) into bacteria, and also into non-animal eukaryotic cells, including plant cells. Transformation is hence the genetic alteration of a bacterial or non-animal eukaryotic cell resulting from the direct uptake through the cell membrane(s) from its surroundings and subsequent incorporation of exogenous genetic material (nucleic acid molecules). Transformation can be affected by artificial means. For transformation to happen, cells or bacteria must be in a state of competence, which might occur as a time-limited response to environmental conditions such as starvation and cell density. For prokaryotic transformation, techniques can include heat shock mediated uptake, bacterial protoplast fusion with intact cells, microinjection and electroporation. Techniques for plant transformation include Agrobacterium mediated transfer, such as by A. tumefaciens, rapidly propelled tungsten or gold microprojectiles, electroporation, microinjection and polyethylene glycol mediated uptake.

In view of the above, this disclosure thus further provides host cells comprising at least one polynucleotide sequence and/or vector as described herein.

For expression of the binding molecule of this disclosure, a host cell may be chosen that modulates the expression of the inserted polynucleotide sequences, and/or modifies and processes the gene product (i.e. RNA and/or protein) as desired. Such modifications (e.g., glycosylation) and processing (e.g., cleavage) of gene products may contribute to the function of the binding molecule. Different host cells have characteristic and specific mechanisms for the post-translational processing and modification of gene products. Appropriate cell lines or host systems can be chosen to ensure the correct modification and processing of the product. To this end, eukaryotic host cells that possess the cellular machinery for proper processing of the primary transcript, glycosylation, and phosphorylation of the gene product may be used.

Exemplary mammalian host cells that can be used for expressing the binding molecules provided herein include Chinese Hamster Ovary (CHO cells) including DHFR minus CHO cells such as DG44 and DUXBl 1 and as described in U.S. Pat. No. 4,634,665 (e.g. used with a DHFR selectable marker, e.g., as described in U.S. Pat. No. 5,179,017), NSO, COS (a derivative of CVI with SV40 T antigen), HEK293 (human kidney), and SP2 (mouse myeloma) cells. Other exemplary host cell lines include, but are not limited to, HELA (human cervical carcinoma), CVI (monkey kidney line), VERY, BHK (baby hamster kidney), MDCK, 293, WI38, R1610 (Chinese hamster fibroblast) BALBC/3T3 (mouse fibroblast). HAK (hamster kidney line), P3×63-Ag3.653 (mouse myeloma), BFA-IcIBPT (bovine endothelial cells), and RAJI (human lymphocyte). Host cell lines may be available from commercial services, the American Tissue Culture Collection or from published literature.

Non-mammalian cells such as bacterial, yeast, insect or plant cells are also readily available and can in principle be used for expression of the binding molecules of this disclosure. Exemplary bacterial host cells include enterobacteriaceae, such as, Escherichia coli, Salmonella; Bacillaceae, such as, Bacillus subtilis; Pneumococcus; Streptococcus; and Haemophilus influenzae.

Other host cells include yeast cells, such as Saccharomyces cerevisiae, and ichiapastoris. Insect cells include, without limitation, Spodopteraflugiperda cells.

In accordance with the foregoing, conceivable expressions systems (i.e., host cells comprising an expression vector) include microorganisms such as bacteria (e.g., E. coli, B. subtilis) transformed with recombinant bacteriophage DNA, plasmid DNA or cosmid DNA expression vectors; yeast (e.g., Saccharomyces, Pichia) transformed with recombinant yeast expression vectors; insect cell systems infected with recombinant virus expression vectors (e.g., baculovirus); plant cell systems infected with recombinant virus expression vectors (e.g., cauliflower mosaic virus, CaMV; tobacco mosaic virus, TMV) or transformed with recombinant plasmid expression vectors (e.g., Ti plasmid); or mammalian cell systems (e.g., COS, CHO, BLK, 293, 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter).

For expression of the binding molecules of this disclosure, eukaryotic cells are preferably envisaged. Accordingly, CHO cells comprising a eukaryotic vector with a polynucleotide sequence encoding the binding molecule of this disclosure (which may for instance be operably linked to the major immediate-early promoter (MIEP) of human cytomegalovirus (CMV)) are useful expression systems for producing the binding molecules of this disclosure.

### Cultivation

The host cells harboring the expression vector are grown under conditions appropriate to the production of the binding molecules described herein, e.g., light chains and heavy chains as described herein, and assayed for heavy and/or light chain protein synthesis. Thus, this disclosure includes host cells containing a polynucleotide encoding a binding molecule of this disclosure, or a heavy or light chain thereof, operably linked to a promoter. For the expression of double-chained antibodies, vectors encoding both the heavy and light chains may be co-expressed in the host cell for expression of the entire molecule.

### Purification

Once a binding molecule of this disclosure has been recombinantly expressed, it may be purified by any purification method known in the art, for example, by chromatography (e.g., ion exchange chromatography (e.g. hydroxylapatite chromatography), affinity chromatography, Protein A. Protein G or lectin affinity chromatography, sizing column chromatography), centrifugation, differential solubility, hydrophobic interaction chromatography, or by any other standard technique for the purification of proteins. The skilled person will readily be able to select a suitable purification method based on the individual characteristics of the binding molecule to be recovered.

In view of the above, this disclosure thus also provides a process for the production of a binding molecule of this disclosure, comprising culturing a host cell as defined herein under conditions allowing the expression of the binding molecule and optionally recovering the produced binding molecule from the culture:

### Pharmaceutical Composition

This disclosure further provides a pharmaceutical composition comprising a therapeutically effective amount of a binding molecule, nucleic acid, vector and/or host cell of this disclosure, and optionally one or more pharmaceutically acceptable excipient(s) or carriers. A preferred pharmaceutical composition comprises an antibody of this disclosure and optionally one or more pharmaceutically acceptable excipient(s).

In one aspect, this disclosure thus relates to a pharmaceutical composition comprising, as an active agent, a binding molecule as described herein, preferably, an anti-FXI antibody or an antigen-binding fragment thereof. Accordingly, the use of the binding molecules for the manufacture of a pharmaceutical composition is also envisaged herein. The term “pharmaceutical composition” preferably refers to a composition suitable for administering to a subject, and more specifically, a human. However, compositions suitable for administration to non-human animals are also encompassed by the term.

The pharmaceutical composition and its components (i.e., active agents and optionally excipients) are preferably pharmaceutically acceptable, i.e., capable of eliciting the desired therapeutic effect without causing undesirable local or systemic effects in the recipient. Pharmaceutically acceptable compositions of this disclosure may be, e.g., sterile and/or pharmaceutically inert. Specifically, the term “pharmaceutically acceptable” may mean approved by a regulatory agency or other generally recognized pharmacopoeia for use in animals, and preferably, in humans.

The binding molecule described herein is preferably present in the pharmaceutical composition in a therapeutically effective amount. By “therapeutically effective amount” is meant an amount or dosage of the binding molecule that elicits the desired therapeutic effect. Therapeutic efficacy and toxicity can be determined by standard pharmaceutical procedures in cell cultures, experimental animals, or clinical trials e.g., ED₅₀ (the dose therapeutically effective in 50% of the population) and LD₅₀ (the dose lethal to 50% of the population). The dose ratio between therapeutic and toxic effects is the therapeutic index, and it can be expressed as the ratio, ED₅₀/LD₅₀. Pharmaceutical compositions that exhibit large therapeutic indices are preferred.

As set out herein, the pharmaceutical composition may optionally comprise one or more excipients and/or additional active agents.

Antibodies and fragments thereof are generally administered parenterally, and preferably intravenously (injection or infusion) or subcutaneously. Compositions for parenteral administration include sterile aqueous or non-aqueous solutions, suspensions and emulsions. Non-aqueous solvents include without limitation, propylene glycol, polyethylene glycol, vegetable oil such as olive oil, and injectable organic esters such as ethyl oleate. Aqueous solvents may be chosen from the group consisting of water, alcohol/aqueous solutions, emulsions or suspensions including saline and buffered media such as without limitation phosphate buffered saline solution. Parenteral vehicles further include sodium chloride solution, Ringer's dextrose, dextrose and sodium chloride, lactated Ringer's, or fixed oils. Other suitable pharmaceutical carriers, diluents and/or excipients are well known in the art. The binding molecule, such as an antibody or antibody fragment thereof, according to this disclosure may be combined with a pharmaceutically acceptable carrier, diluent and/or excipient such as those discussed above to form a pharmaceutical composition. The pharmaceutical compositions may comprise the binding molecule of this disclosure in an aqueous carrier that comprises a buffering agent selected from the group consisting of a histidine buffer, acetic acid buffer, citric acid buffer, and a histidine/HCl buffer. Additional buffers and formulation information is available to one of skill in the art, for example in Wang W et al., J. Pharmaceutical Sci. 2007 January (1):1-26. Preservatives may also be present such as, for example, antimicrobials, anti-oxidants, chelating agents, inert gases, etc.

The pharmaceutical composition may further comprise proteinaceous carriers such as, for example, serum albumin or immunoglobulin, preferably of human origin. In one embodiment, the pharmaceutical composition comprises the binding molecule in lyophilized form, and preferably is reconstituted in solution or suspension prior to administration. In another embodiments, the pharmaceutical composition comprises the binding molecule and is in liquid form.

After pharmaceutical compositions of this disclosure and optionally a suitable excipient have been prepared, they can be placed in an appropriate container and labeled for treatment of an indicated condition. Such labeling would for instance include amount, frequency and method of administration.

### Additional Active Agents

This disclosure further provides medicaments or pharmaceutical compositions comprising an inventive compound and one or more further active ingredients, including for treatment and/or prophylaxis of the disorders mentioned herein. Preferred examples of active ingredients suitable for combinations include but are not limited to:
lipid-lowering substances, especially HMG-CoA (3-hydroxy-3-methylglutaryl-coenzyme A) reductase inhibitors, including without limitation lovastatin (Mevacor), simvastatin (Zocor), pravastatin (Pravachol), fluvastatin (Lescol) and atorvastatin (Lipitor);
coronary therapeutics/vasodilatators, especially ACE (angiotensin converting enzyme) inhibitors, including without limitation captopril, lisinopril, enalapril, ramipril, cilazapril, benazepril, fosinopril, quinapril and perindopril, or All (angiotensin II) receptor antagonists, including without limitation embusartan, losartan, valsartan, irbesartan, candesartan, eprosartan and temisartan, or adrenoceptor antagonists, including without limitation carvedilol, alprenolol, bisoprolol, acebutolol, atenolol, betaxolol, carteolol, metoprolol, nadolol, penbutolol, pindolol, propanolol and timolol, or alpha-1-adrenoceptor antagonists, including without limitation prazosine, bunazosine, doxazosine and terazosine, or diuretics, including without limitation hydrochlorothiazide, furosemide, bumetanide, piretanide, torasemide, amiloride and dihydralazine, or calcium channel blockers, including without limitation verapamil and diltiazem, or dihydropyridine derivatives, including without limitation nifedipin (Adalat) and nitrendipine (Bayotensin), or nitro preparations, including without limitation isosorbide 5-mononitrate, isosorbidedinitrate and glycerol trinitrate, or substances causing an increase in cyclic guanosine monophosphate (cGMP), including without limitation stimulators of soluble guanylatecyclase, including without limitation riociguat;
plasminogen activators (thrombolytics/fibrinolytics) and compounds which promote thrombolysis/fibrinolysis including without limitation inhibitors of the plasminogen activator inhibitor (PAI inhibitors) or inhibitors of the thrombin-activated fibrinolysis inhibitor (TAFI inhibitors), including without limitation tissue plasminogen activator (t-PA), streptokinase, reteplase and urokinase;
anticoagulatory substances (anticoagulants), including without limitation heparin. (UFH), low-molecular-weight heparins (LMW), including without limitation tinzaparin, certoparin, parnaparin, nadroparin, ardeparin, enoxaparin, reviparin, dalteparin, danaparoid, semuloparin (AVE 5026), adomiparin (M118) and EP-42675/ORG42675;
direct thrombin inhibitors (DTI), including without limitation Pradaxa (dabigatran), atecegatran (AZD-0837), DP-4088, SSR-182289A, argatroban, bivalirudin and tanogitran (BIBT-986 and prodrug BIBT-1011), hirudin;
direct factor Xa inhibitors including without limitation, rivaroxaban, apixaban, edoxaban (DU-176b), betrixaban (PRT-54021), R-1663, darexaban (YM-150), otamixaban (FXV-673/RPR-130673), letaxaban (TAK-442), razaxaban (DPC-906), DX-9065a, LY-517717, tanogitran (BIBT-986, prodrug: BIBT-1011), idraparinux and fondaparinux,
platelet aggregation-inhibiting substances (platelet aggregation inhibitors, thrombocyte aggregation inhibitors), including without limitation acetylsalicylic acid (for example Aspirin), ticlopidine (Ticlid), clopidogrel (Plavix), prasugrel, ticagrelor, cangrelor, elinogrel, vorapaxar;
fibrinogen receptor antagonists (glycoprotein-IIb/IIIa antagonists), including without limitation abciximab, eptifibatide, tirofiban, lamifiban, lefradafiban and fradafiban;
antiarrhythmics;
various antibiotics or antifungal medicaments, either as calculated therapy (prior to the presence of the microbial diagnosis) or as specific therapy;
vasopressors, including without limitation norepinephrine, dopamine and vasopressin,
inotropic therapy, including without limitation dobutamine;
recombinant human activated protein C, for example Xigris;
blood products, including without limitation erythrocyte concentrates, thrombocyte concentrates, erythropietin and fresh frozen plasma;
inhibitors of platelet adhesion like GPV1 and/or GPIb antagonists including without limitation Revacept or Caplacizumab;
inhibitors of the VEGF and/or PDGF dependent signal transduction pathways including without limitation Afiibercept, Ranibizumab, Bevacizumab, KH-902, Pegaptanib, Ramucirumab, Squalaminoder Bevasiranib, Apatinib, Axitinib. Brivanib, Cediranib, Dovitinib, Lenvatinib, Linifanib, Motesanib, Pazopanib, Regorafenib, Sorafenib, Sunitinib, Tivozanib, Vandetanib, Vatalanib, Vargatef or E-10030;
inhibitors of the Angiopoietin-Tie signal transduction pathway including without limitation AMG386;
inhibitors of the Tie2 receptor tyrosine kinase activity;
inhibitors of the Integrine dependent signal transduction pathways including without limitation Volociximab, Cilengitid or ALG1001;
inhibitors of the PI3Kinase-AKT-mTor dependent signal transduction including without limitation XL-147, Perifosin, MK2206, Sirolimus, Temsirolimus or Everolimus;
Corticosteroids including without limitation hydrocortisone, fludrocortisone, Anecortave, Betamethason, Dexamethason, Triamcinolon, Fluocinolon or Fluocinolonacetonid;
inhibitors of the ALK1-Smad1/5 dependent signal transduction pathway including without limitation ACE0411;
inhibitors of Cyclooxygeneases including without limitation Bromfenac or Nepafenac;
inhibitors of the Kallikrein-Kinin system including without limitation Safotibant or Ecallantid;
inhibitors of the Sphingosin-1-phosphat dependent signal transduction pathways including without limitation Sonepcizumab;
inhibitors of the Complement C5a receptor including without limitation Eculizumab;
inhibitors of the 5HT1a receptor including without limitation Tandospiron;
inhibitors of the Raf-Mek-Erk dependent signal transduction pathway, inhibitors of the MAPK signal transduction pathway; inhibitors of the FGF signal transduction pathway, inhibitors of endothelial cell proliferation; and compounds which are able to induce apoptosis; or
Photodynamic therapies, consisting of an active substance and the exposure to light, whereas the active substance is for example Verteporfin.

“Combinations” for the purpose of this disclosure mean not only dosage forms which contain all the components (so-called fixed combinations) and combination packs which contain the components separate from one another, but also components which are administered simultaneously or sequentially, provided that they are used for prophylaxis and/or treatment of the same disease. It is likewise possible to combine two or more active ingredients with one another, meaning that they are thus each in two-component or multicomponent combinations.

### Administration

A variety of routes are applicable for administration of the pharmaceutical composition according to this disclosure. Administration may be accomplished parentally. Methods of parenteral delivery include, for example, topical, intra-arterial, intramuscular, subcutaneous, intramedullary, intrathecal, intraventricular, intravenous, intraperitoneal, intrauterine, intravaginal, sublingual or intranasal administration.

Pharmaceutical formulations for parenteral administration include aqueous solutions of active compounds. For injection, the pharmaceutical compositions of this disclosure may be formulated in aqueous solutions, preferably in physiologically compatible buffers such as Hank's solution, Ringer's solution, or physiologically buffered saline. Aqueous injection suspensions may contain substances that increase viscosity of the suspension, such as sodium carboxymethyl cellulose, sorbitol, or dextran. Additionally, suspensions of the active compounds may be prepared as appropriate oily injection suspensions. Exemplary lipophilic solvents or vehicles include fatty oils such as sesame oil, or synthetic fatty acid esters, such as ethyl oleate or triglycerides, or liposomes. Optionally, the suspension may also contain suitable stabilizers or agents which may increase the solubility of the compounds to allow for the preparation of highly concentrated solutions. For topical or nasal administration, penetrants appropriate to the barrier to be permeated are used in the formulation. Such penetrants are generally known in the art. Further details on techniques for formulation and administration may be found in the 22nd edition of Remington's Pharmaceutical Sciences (Ed. Mack Publishing Co, Easton, Pa., 2012).

### Treatment

As used herein, the terms “treat” or “treatment” (in all grammatical forms) include therapeutic or prophylactic treatment of the diseases described herein. A “therapeutic or prophylactic treatment” comprises prophylactic treatments aimed at the slowing of or complete prevention of clinical and/or pathological manifestations or therapeutic treatment aimed at amelioration or remission of clinical and/or pathological manifestations. The term “treatment” thus also includes the amelioration or prevention of the described diseases. Treatment can also mean prolonging survival as compared to expected survival without treatment. Those in need of treatment include those already with the condition or disorder, those prone to have the condition or disorder, or those in which the condition or disorder is to be prevented.

The terms “subject” or “individual” or “animal” or “patient” or “mammal” are used interchangeably herein to refer to any subject, preferably a mammalian subject, for whom diagnosis, prognosis, or treatment (therapy) is desired. Mammalian subjects include humans, non-human primates, domestic animals, farm animals, companion animals, and zoo, sports, or pet animals, such as, dogs, cats, guinea pigs, rabbits, rats, mice, horses, cattle, cows, and the like.

As used herein, phrases regarding a subject such as “would benefit from” and “in need of treatment” include subjects that would benefit from administration of the humanized monoclonal antibody, binding-fragment, variant, or derivative thereof.

### Dosage

The exact dosage (therapeutically effective amount) of the binding molecule, polynucleotide, vector, or host cell will be ascertainable by one skilled in the art using known techniques. Suitable dosages provide sufficient amounts of the binding molecule and are preferably therapeutically effective, i.e., elicit the desired therapeutic or prophylactic effect.

As is known in the art, determination of and adjustments for purpose of the treatment (e.g., prophylaxis, remission maintenance, acute flare of disease), route, time and frequency of administration, time and frequency of administration formulation, age, body weight, general health, sex, diet, severity of the disease state, drug combination(s), reaction sensitivities, and tolerance/response to therapy may be made. Suitable therapeutically effective dosage ranges can be determined using data obtained from cell culture assays and animal studies, and may include the ED₅₀. Dosage amounts may vary from 0.1 to 100000 micrograms, up to a total dose of about 2 g, depending upon the route of administration. Exemplary dosages of the binding molecule may range from about 0.01 mg/kg to about 10 mg/kg, from about 0.1 mg/kg to about 10 mg/kg, from about 1 mg/kg to about 10 mg/kg, from about 1 mg/kg to about 5 mg/kg, from about 0.01 mg/kg to about 1 mg/kg, or from about 0.1 mg/kg to about 1 mg/kg. Guidance as to dosages and methods of deliver), is provided in the literature. It is recognized that treatment may require a single administration of a therapeutically effective dose or multiple administrations of a therapeutically effective dose of the binding molecule, polynucleotide, vector, or host cell of this disclosure. For example, some pharmaceutical compositions might be administered once, periodically over a certain hourly time period, every 3 to 4 days, every week, or once every two weeks, once within a month, once within two months, depending on formulation, half-life and clearance rate of the formulation. The determination of each of the applicable variables is well known by the skilled artisan.

### Kit

This disclosure further relates to pharmaceutical packs and kits comprising one or more containers or vials filled with one or more of the active agents of the aforementioned pharmaceutical compositions of this disclosure, along with instructions for the administration thereof. Thus, also provided herein is a kit comprising a binding molecule, a polynucleotide, a vector, a host cell, and/or the pharmaceutical composition as described herein. The aforementioned kits described herein may be used for treatment of the diseases set out herein, or for other purposes.

Associated with the aforementioned container(s) can be a notice in the form prescribed by a governmental agency regulating the manufacture, use, or sale of pharmaceuticals or biological products, reflecting approval by the agency of the manufacture, use, or sale of the product for human administration.

The kit may comprise one or more active agents (optionally formulated as a pharmaceutical composition with one or more excipients). Suitable active agents have previously been listed in the context of the pharmaceutical composition and are also conceivable as parts of the inventive kit. The additional active agent can be administered simultaneously or sequentially with respect to the binding molecule, nucleic acid sequence, vector, host cell, and/or the pharmaceutical composition to the patient. This disclosure further encompasses the administration of the active agents via different routes, e.g., orally and intravenously.

Further envisaged herein are kits comprising polynucleotide sequences encoding the binding molecules of this disclosure. The polynucleotides may be provided in a vector, such as a plasmid, suitable for transfection into and expression by a host cell. Such vectors and host cells are described herein.

### Therapeutic Use

This disclosure further provides binding molecules of this invention, preferably antibodies and antigen-binding fragments thereof, for use as medicaments for the treatment and/or prophylaxis of diseases in humans and/or animals.

This disclosure further provides binding molecules of this disclosure, preferably antibodies and antigen-binding fragments thereof, for use in the treatment and/or prophylaxis of disorders, e.g., cardiovascular disorders, preferably thrombotic or thromboembolic disorders and/or thrombotic or thromboembolic complications, and inflammatory conditions, preferably autoimmune inflammations or infection-associated inflammatory responses.

FXIa is an enzyme that, in the context of coagulation, can be activated both by thrombin and FXIIa, and is therefore involved in processes of coagulation. FXI is a central component of the transition from initiation to amplification and propagation of coagulation. Moreover, FXIa is a component fir the initiation and maintenance of pathological blood coagulation inside blood vessels. The contact activation system of blood can become activated on negatively charged intravascular surfaces, which include not only exposure of flowing blood to subendothelial or extravascular matter, including collagen and laminin, exposure of surface structures of foreign cells (e.g., bacteria), but also artificial surfaces such as vascular prostheses, catheters, stents, ventricular assist devices, valves, and extracorporeal life support systems, such as oxygenators, pumps, tubing, dialyzers, and alike. On the surface, factor XII (FXII) is activated to factor XIIa (FXIIa) which subsequently activates FXI that is attached to surfaces to FXIa. FXI can also become autoactivated on negatively charged surfaces. This FM activation leads to downstream thrombin generation, as well as feedback amplification of all contact activation complex enzymes comprising FXI, FXII and prekallikrein.

In contrast, hemostatic thrombin generation outside blood vessels in blood that escapes the vessel through a wound remains uninfluenced by inhibition of contact activation since hemostatic thrombin generation is driven by the TF/FVIIa complex and feedback activation of FXI by thrombin, neither of which are significantly affected by pharmacological interference with functions of the contact activation complex. The absence of demonstrable bleeding tendency and reduced propensity for acute inflammation in contact system-compromised mammals, which characterizes the in vivo effect of complex formation between FXI and the binding molecule, is of great advantage versus other types of FXI/FXIa inhibitors or other protease inhibitors for use in humans, e.g., in patients with increased risk of bleeding or inflammatory reactions. Accordingly, the binding molecules of this disclosure, preferably antibodies and antigen-binding fragments thereof, are for use in the treatment and/or prophylaxis of disorders or complications which may arise from the enzymatic and non-enzymatic activities of the contact activation complex, including, among others, pathological contact-initiated thrombin and bradykinin generation that result in thrombus formation and inflammation, respectively.

For the purpose of this disclosure, the “thrombotic or thromboembolic disorders” include, for example, disorders which occur both in the arterial and in the venous vasculature and which can be treated with the binding molecules of this disclosure, preferably antibodies and antigen-binding fragments thereof, and preferably, disorders in the coronary arteries of the heart, such as, acute coronary syndrome (ACS), myocardial infarction with ST segment elevation (STEMI) and without ST segment elevation (non-STEMI), stable angina pectoris, unstable angina pectoris, reocclusions and restenoses after coronary interventions such as angioplasty, stent implantation or aortocoronary bypass, but also thrombotic or thromboembolic disorders in further vessels leading to peripheral arterial occlusive disorders, pulmonary embolisms, venous thromboembolisms, venous thromboses, i.e., in deep leg veins and kidney veins, transitory ischemic attacks and also thrombotic stroke and thromboembolic stroke.

For the purposes of this disclosure, the “inflammatory disorders” include all assembled contact system complex (FXII/FXI/PK/HK) activation-supported or -mediated pathological events, including excessive cleavage of HK (HMWK) and generation of the most potent known proinflammatory peptide, bradykinin, with consequential or related pathological vasodilation and increased blood vessel permeability, blood pressure dysregulation, increased immune responses for foreign substances and excessive endogenous autoimmune responses, including those that involve antigen-antibody reactions and therefore complement and plasminogen activation, and other consequential events that affect either the local environment (cells, organs) or the entire body system.

Activation of the contact system may occur by various causes or associated disorders. In the context of surgical interventions and other tissue traumas, immobility, confinement to bed, infections, inflammation, cancer, tissue injury and necrosis, autoimmunity, temporal or chronic implanted foreign bodies, and ischemia, inter alia, the contact system can be activated causing thrombotic and inflammatory complications. The binding molecules of this disclosure, preferably antibodies and antigen-binding fragments thereof, are therefore useful in the prophylaxis of thrombosis and inflammation in the context of surgical interventions, for example, in patients undergoing gastrointestinal, pulmonary, nervous system, urological, orthopedic, and other major surgeries known or having potential to be associated with thrombus formation and/or inflammation. The binding molecules of this disclosure, preferably antibodies and antigen-binding fragments thereof, are therefore also for use in the prophylaxis of thrombosis in patients having an activated contact system.

The binding molecules of this disclosure, preferably antibodies and antigen-binding fragments thereof, are therefore also for use in the treatment and/or prophylaxis of venous and cardiogenic thromboembolisms, e.g., brain ischemia, stroke and systemic thromboembolisms and ischemia, in patients with acute, intermittent or persistent cardiac arrhythmias, e.g., atrial fibrillation, in patients undergoing cardioversion, and also in patients with heart valve disorders or with artificial heart valves. In addition, the binding molecules of this disclosure, preferably antibodies and antigen-binding fragments thereof, are for use in the treatment and/or prophylaxis of disseminated intravascular coagulation (DIC) which may occur in connection with sepsis, inter alia, but also owing to surgical interventions, neoplastic disorders, hums or other injuries and may lead to severe organ damage through microthromboses.

Thromboembolic and inflammatory complications furthermore occur in microangiopathic hemolytic anemias and by the blood coming into contact with foreign surfaces in the context of extracorporeal circulation (e.g., cardiopulmonary bypass), other life support systems such as hemodialysis, extracorporeal membrane oxygenation (ECMO), left ventricular assist device (LVAD), and similar methods, AV fistulas, vascular and heart valve prostheses.

Moreover, the binding molecules of this disclosure, preferably antibodies and antigen-binding fragments thereof, are for use in the treatment and/or prophylaxis of disorders involving microdot formation or fibrin deposits in cerebral blood vessels which may lead to dementia disorders such as vascular dementia or Alzheimer's disease. Here, the clot may contribute to the disorder both via occlusions and by binding further disease-relevant factors.

Moreover, the binding molecules of this disclosure, preferably antibodies and antigen-binding fragments thereof, can be used for the prophylaxis and/or treatment of thrombotic and/or thromboembolic complications, venous thromboembolisms in cancer patients, including those undergoing major surgical interventions or chemo- or radiotherapy.

In the context of this disclosure, the term “pulmonary hypertension” includes pulmonary arterial hypertension, pulmonary hypertension associated with disorders of the left heart, pulmonary hypertension associated with pulmonary disorders and/or hypoxia and pulmonary hypertension owing to chronic thromboembolisms (CUPID.

In addition, the binding molecules of this disclosure, preferably antibodies and antigen-binding fragments thereof; are also for use in the treatment and/or prophylaxis of systemic inflammation and disseminated intravascular coagulation (DIC) in the context of an infectious disease, and/or of systemic inflammatory response syndrome (SIRS), septic organ dysfunction, septic organ failure and multiorgan failure, acute respiratory distress syndrome (ARDS), acute lung injury (ALI), septic shock and/or septic organ failure. In the course of an infection, there may be a generalized activation of the contact and coagulation systems, causing symptomatic DIC (with or without consumptive coagulopathy), with (micro)thrombosis in various organs and secondary hemorrhagic complications. Moreover, there may be endothelial damage with increased permeability of the vessels and diffusion of fluid and proteins into the extravasal space. As the infection progresses, there may be failure of an organ (for example kidney failure, liver failure, respiratory failure, central-nervous deficits and cardiovascular failure, etc.) or multiorgan failure. In the case of DIC, there is a massive activation of the coagulation system at the surface of damaged endothelial cells, the surfaces of foreign bodies or crosslinked extravascular tissue. As a consequence, there is coagulation in small vessels of various organs with hypoxia and subsequent organ dysfunction. A secondary effect is the consumption of coagulation factors (consumptive coagulopathy), for example protein C, protein S, protein Z, FII, FV, FVIII, FIX, FX, FXI, FXII, FXIII, and fibrinogen (FI) and platelets, which reduces the control of blood homeostatic balance and may result in heavy and even fatal bleeding.

The binding molecules of this disclosure, preferably antibodies and antigen-binding fragments thereof, are also for use in the primary prophylaxis of thrombotic or thromboembolic disorders and/or inflammatory disorders and/or disorders with increased vascular permeability in patients in which gene mutations lead to enhanced activity of the enzymes, or increased levels of the zymogens and these are established by relevant tests/measurements of the enzyme activity or zymogen concentrations.

In addition, the binding molecules of this disclosure, preferably antibodies and antigen-binding fragments thereof can also be used for preventing coagulation ex vivo, for example, for the protection of organs to be transplanted against organ damage caused by formation of clots and for protecting the organ recipient against thromboemboli from the transplanted organ, for preserving blood and plasma products, for cleaning/pretreating catheters and other medical auxiliaries and instruments, for coating synthetic surfaces of medical auxiliaries and instruments used in vivo or ex vivo or for biological samples which may comprise FXI/FXIa.

This disclosure further provides for the use of the binding molecules of this disclosure, preferably antibodies and antigen-binding fragments thereof, for the treatment and/or prophylaxis of disorders, especially the disorders mentioned above.

This disclosure further provides for the use of the binding molecules of this disclosure, preferably antibodies and antigen-binding fragments thereof, for production of a medicament for the treatment and/or prophylaxis of disorders, especially the disorders mentioned herein, preferably for producing a medicament for the treatment and/or prophylaxis of thrombotic or thromboembolic disorders.

This disclosure further provides a method for the treatment and/or prophylaxis of disorders, especially the disorders mentioned herein, using a therapeutically effective amount of a binding molecule of this disclosure, preferably an antibody and antigen-binding fragment thereof.

This disclosure further provides the binding molecules of this disclosure, preferably antibodies and antigen-binding fragments thereof, for use in a method for the treatment and/or prophylaxis of disorders, especially the disorders mentioned herein, using a therapeutically effective amount of a binding molecule of this disclosure, preferably antibody and antigen-binding fragment thereof.

This disclosure further provides methods of treatment of thrombotic or thromboembolic disorders in man and/or animals by administration of a therapeutically effective amount of at least one binding molecule of this disclosure, preferably antibody and antigen-binding fragment thereof or a pharmaceutical composition of this disclosure. This disclosure further provides a method of inhibiting blood coagulation, platelet aggregation and/or thrombosis in a subject by administration of a therapeutically effective amount of at least one binding molecule of this disclosure, preferably antibody and antigen-binding fragment thereof or a pharmaceutical composition of this disclosure.

### Gene Therapy

Further provided herein is a transfer vector for use in mammalian gene therapy that comprises a polynucleotide as disclosed herein, and methods of treating or preventing disease comprising incorporating exogenous nucleic acid as described herein into the cell of a mammalian patient in need thereof, such that the exogenous nucleic acid is expressed and the disease is prevented or treated. In one embodiment, nucleic acid molecules encoding both a heavy chain and a light chain are administered to a patient. In a preferred embodiment, the nucleic acid molecules are administered such that they are stably integrated into chromosomes of B cells because these cells are specialized for producing antibodies. In one embodiment, precursor B cells are transfected or infected ex vivo and re-transplanted into a patient in need thereof. In another embodiment, precursor B cells or other cells are infected in vivo using a recombinant virus known to infect the cell type of interest.

In a preferred embodiment, the gene therapy method comprises administering an isolated nucleic acid molecule encoding the heavy chain or an antigen-binding portion thereof of an anti-FYI antibody as disclosed herein and expressing the nucleic acid molecule. In another preferred embodiment, the gene therapy method comprises administering an isolated nucleic acid molecule encoding the light chain or an antigen-binding portion thereof of an anti-FXI antibody as disclosed herein and expressing the nucleic acid molecule. In another embodiment, the gene therapy method comprises administering an isolated nucleic acid molecule encoding the heavy chain or an antigen-binding portion thereof and an isolated nucleic acid molecule encoding the light chain or the antigen-binding portion thereof of an anti-FXI antibody as disclosed herein and expressing the nucleic acid molecule.

Specific conditions for the uptake of exogeneous nucleic acid are well known in the art. They include, but are not limited to, retroviral infection, adenoviral infection, transformation with plasmids, transformation with liposomes containing exogeneous nucleic acid, biolistic nucleic acid delivery (i.e., loading the nucleic acid onto gold or other metal particles and shooting or injecting into the cells), adeno-associated virus infection and Epstein-Barr virus infection. These may all be considered “expression vectors” for the purposes of this disclosure. The expression vectors may be either extrachromosomal vectors or vectors which integrate into a host genome. Generally, these expression vectors include transcriptional and translational regulatory nucleic acid operably linked to the exogeneous nucleic acid. In general, the transcriptional and translational regulatory sequences may include but are not limited to, promoter sequences, ribosomal binding sites, transcriptional start and stop sequences, translational start and stop sequences, and enhancer or activator sequences. In a preferred embodiment, the regulatory sequences include a promoter and transcriptional start and stop sequences. In addition, the expression vector may comprise additional elements. For example, for integrating expression vectors, the expression vector contains at least one sequence homologous to the host cell genome, and preferably two homologous sequences which flank the expression construct. The integrating vector may be directed to a specific locus in the host cell by selecting the appropriate homologous sequence for inclusion in the vector. Constructs for integrating vectors are well known in the art.

### EXPERIMENTS

Starting front the murine 14E11 anti-FXI antibody, novel humanized antibodies were generated, Candidate antibodies were tested for their FXI binding activity determined by competition ELISA and by their ability to inhibit the conversion of FXI into its active form. Exemplary antibody, AB023, was characterized. Studies were performed using AB023 to confirm that the binding and anticoagulant properties were maintained and comparable after humanization of 14E11 by CDR-grafting. In vitro binding studies showed that AB023 binds with high affinity to both mouse and human FXI (0.16 nM and 3.2 nM respectively), however, with lower affinity than the mouse monoclonal antibody 14E11. In vitro aPTT assays confirmed that the anticoagulant effect was maintained after humanization as AB023 prolonged the aPTT in human, baboon, cynomolgus monkey, and rat plasma in a concentration-dependent manner.

Interestingly, some differences between 14E11 and AB023 were found in several in vitro assays. First, AB023 was able to inhibit FXI autoactivation in the presence of both dextran sulfate and DNA in a concentration-dependent manner, whereas 14E11 was unable to do so at all concentrations tested. Second, AB023 inhibited activation of FXII by FXIa in a concentration dependent manner in contrast to 14E11. And finally, AB023 was more effective at inhibiting human FXIIa activation of human FXI in the presence HK and dextran sulfate.

The antithrombotic effects seen with 14E11 (Cheng Q, Tucker E I, Pine M S, Sisler I, Matafonov A, Sun M F, White-Adams T C, Smith S A. Hanson S R, McCarty O J, Renné T, Gruber A, Gailani D. Blood. 2010 Nov. 11; 116(19):3981-3989; Tucker E I, Verbout N G, Leung P Y, Hurst S, McCarty O J, Gailani D, Gruber A. Blood. 2012 May 17; 119(20):4762-8) were maintained after humanization (AB023) as demonstrated in both the mouse model of arterial thrombosis and the baboon model of arterial and venous thrombosis. In the mouse model of arterial thrombosis, AB023 prevented FeCl₃-induced carotid artery occlusion comparable to that of total FXI deficiency, however, this effect was not as great as the effect produced by the murine monoclonal antibody (14E11) at the same dose. In the well-established baboon thrombosis model using a thrombogenic graft plus expansion chamber to mimic arterial-type and venous-type flow respectively, administration of a low dose of AB023 (0.2 mg/kg, i.v.) slightly reduced platelet accumulation rates within the vascular grafts compared to controls while near complete inhibition of platelet accumulation was achieved within the expansion chamber. Fibrin deposition was also lower in both arterial-type and venous-type thrombosis. Interestingly, using a thrombogenic graft without an expansion chamber, 1.0 mg/kg AB023, i.v. reduced both platelet and fibrin deposition within the vascular graft segment itself, as well as prevented formation of the downstream thrombus “tail,” demonstrating that AB023, like its murine precursor, 14E11, prevented venous-type thrombosis at all doses tested. In addition, this study suggests that AB023 also appeared to reduce arterial-type thrombosis at higher doses as evidenced by the reduction of platelets and fibrin in collagen coated thrombogenic graft.

The anti-inflammatory effects of AB023 were tested in a baboon model of sepsis and where as 14E11 was previously tested infection models in mice (Silasi R et al., Inhibition of contact-mediated activation of factor XI protects baboons against S aureus-induced organ damage and death. Blood Advances 2019 3:658-669, data not shown; Tucker E I et al., Inhibition of factor XI activation attenuates inflammation and coagulopathy while improving the survival of mouse polymicrobial sepsis. Blood. 2012 May 17; 119(20):4762-8.). All tests that were used to compare the activity of AB023 to 14E11 indicated equivalence of the effects. The anticoagulant effect of AB023 was also evaluated in healthy human subjects (Lorentz C U, et al., Contact Activation Inhibitor and Factor XI Antibody, AB023, Produces Safe, Dose-Dependent Anticoagulation in a Phase 1 First-In-Human Trial. Arterioscler Thromb Vasc Biol. 2019 April; 39(4):799-809) and the data (not shown) indicated that AB023 was safe and generated dose-dependent anti-coagulant effect.

Potential cross-reactivity of AB023 was evaluated using cryosections of healthy human tissues and none was found.

Taken together, the studies performed on AB023 demonstrate the anticoagulant and antithrombotic properties of AB023. Additional studies demonstrated a low risk for hemostasis impairment, low probability of cross reactivity and off target effects, low probability for immune activation and immunogenicity, and a low probability of neuro- and cardiotoxic effects (data not shown).

### Characterization of the Murine Antibody 14E11

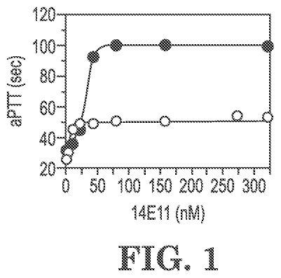

As set forth in the 14E11 Patents and other publications cited herein, studies performed determined the binding and anticoagulant properties of the monoclonal, murine, anti-FXI antibody, 14E11. Briefly, binding and anticoagulant properties were examined in vitro. Results showed that 14E11 prolonged the aPTT of mouse (◯) or human (●) human plasma (FIG. 1) in an aPTT assay, with maximum inhibition at 25-50 nM, which is in the range of FXI concentration in human plasma (20-45 nM). The prolongation of the clotting times was about half of those of FXI deficient human and mouse plasmas. 14E11 did not affect the prothrombin time of human plasma (data not shown).

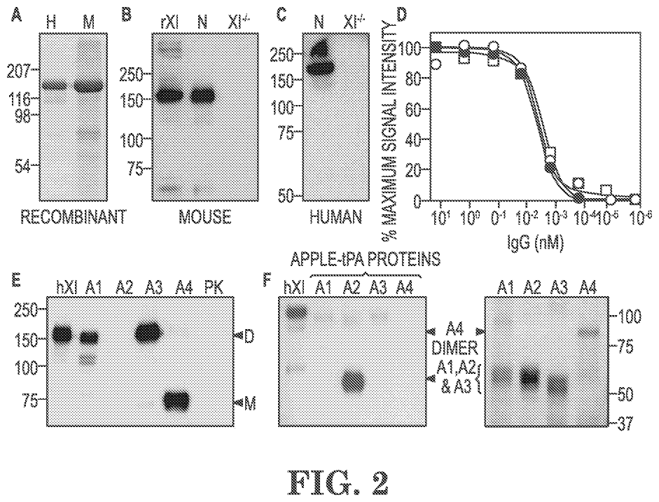

14E11 was found to bind and recognize a single band that is the expected size of the FXI homodimer (˜160 kDa) in normal mouse and human plasma, as well as of recombinant mouse FXI (FIGS. 2A-C) plasma, but no binding was seen in FXI deficient plasma. In a solid phase binding assay, the binding affinity of 14E11 for mouse and human FXI, and human FXIa, was determined (apparent K_{d}˜2-3 pM, FIG. 2D). In most mammals FXI is a homodimer of two 80 kDa subunits, each containing four apple domains (A1-A4) and a protease domain. Prekallikrein (PK), a monomeric homolog of FXI, has a near identical structure to the FXI monomer. Human FXI/PK chimeras were used to show that the A2 domain is required for 14E11 binding to FXI (FIG. 2E). Western blots using individual FXI apple domains linked to tPA indicate the 14E11 binding site is likely located entirely within the A2 domain (FIG. 2F).

As shown, the murine monoclonal antibody 14E11 binds to the apple 2 (A2) domain of human and mouse FXI and inhibits activation of FXI by FXIIa and downstream thrombin generation as well as FXI autoactivation. 14E11 binds to FXI from many different species and can prolong the aPTT in plasma from mouse, human, baboon, rabbit, rat, pig, and rhesus macaque (data not shown).

### Humanization of Murine Antibody 14E11

Humanization of the murine monoclonal antibody 14E11 was performed at Abzena (f.k.a., Antitope Limited, Cambridge, GB) using complementarily determining region (CDR) grafting technology (O'Brien S. and Jones T. 2001. Humanising Antibodies by CDR Grafting. In: Kontermann R., Dübel S. (Eds) Antibody Engineering. Pp, 567-590. Springer Lab Manuals. Springer, Berlin, Heidelberg). In detail, for the selection of germline acceptor family subsets, CDR residues of the murine antibody were determined and annotated following the Kabat numbering system (for details see http://www.bioinf.org.uk/abs/#kabatnum). The canonical structures of the heavy and light chain CDRs were determined using human germline genes in a CDR homology-based approach to antibody humanization, and human germline framework acceptors with the same canonical structures were selected (O'Brien and Jones, 2001: Hwang, 2005). Human germline framework acceptors with the same canonical structures were selected.

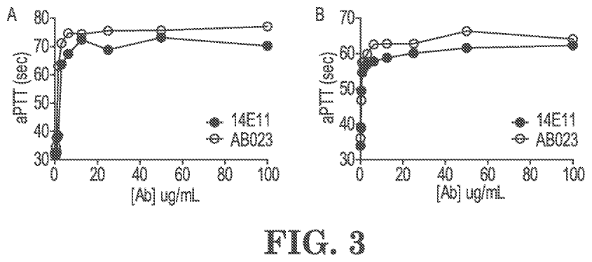

The 14E11 variable (V) region genes were sequenced from RNA isolated from the 14E11 hybridoma cell line and these sequences were used to generate a chimeric antibody and design a series of germline humanized antibody variants. Amino acids in the variable region frameworks of 14E11 that may support the binding properties of the antibody were identified by generating in silico structural models of a chimeric antibody consisting of the 14E11 variable domains and human IgG4 and kappa light chains using Swiss PDB and were noted for incorporation into one or more of the CDR-grafted variants. Humanized V region genes were designed based on human germline sequences with the closest homology to the murine sequences and were constructed by gene synthesis. The humanized heavy chain variable region (VH) variants were then cloned into a first vector containing the human IgG4 heavy chain common regions genes (CH) 1-3 with a modified S241P hinge region using the restriction enzymes Hind III and Mlu I. The humanized light chain variable region variants (Vκ) were cloned into a second vector containing the human kappa common region gene (Cκ) using the restriction enzymes BssH I and BamH I. The chimeric antibody was also created by cloning the murine variable regions into the same vectors. The humanized and chimeric antibodies were stably expressed in NSO cells and tested for binding to the target antigen (recombinant human FXI) in a competition ELISA compared to 14E11 (data not shown). In addition, the anticoagulant properties of the humanized antibodies were tested using the aPTT assay (FIG. 3). The results from these assays revealed the variant VH3/Vκ3 (clone 3G3) as a preferred antibody candidate, AB023, and a stable manufacturing cell line was generated at Abzena using composite CHO™ technology.

Cells from the stable cell line may be used to inoculate a production bioreactor. For example, cells may be cultured using a batch-fed process and harvested, e.g., at 14 days. Cells may then be purified in one or more chromatography column steps, viral clearance steps, and concentrated and diafiltrated. After final formulation in a buffer, the antibody, i.e., AB023, may be, but is not necessarily, filtered and stored frozen as a lyophilized product (e.g., 15 mg/mL after reconstitution).

### Characterization of AB023

AB023 was characterized using a variety of analytical methods. These methods were used to understand the primary structure, partial conformational structure, binding and post-translational modifications of the protein. The analyses used were peptide mapping, mass-spectrometry (MS), chip based capillary electrophoresis, capillary isoelectric focusing (cIEF), size-exclusion (SEC) chromatography, oligosaccharide mapping, fluorescence, circular dichroism (CD) and differential scanning calorimetry (DSC), each of which is well known in the art.

The molecular weight of AB023, 146,560 Da was determined by MS to verify structure and post-translational modifications and establish the intactness of the molecule. The molecular weights of heavy and light chains (49,890 Da and 23.401 Da respectively) were determined after reducing the disulfide bonds in the molecule. Differences compared to the theoretical molecular weight (144,020 Da) are due to post-translational modifications, specifically, glycosylation.

### Binding Affinity

The binding affinity of the humanized monoclonal antibody AB023 for human and mouse coagulation factor XI (FXI) was determined using a solid phase binding assay. In addition, the binding affinity of AB023 to activated human FXI (FXIa) for both A13023 and 14E11 was evaluated. Briefly, for this solid phase binding assay both 14E11 and AB023 were biotinylated using the EZ-Link™ Sulfo-NHS-Biotinylation Kit (ThermoFisher Scientific, Waltham, Mass.) as per instructions. Microtiter plates were coated with FXI or FXIa (2 μg/ml, 100 μL/well) in 50 mmol/L Na₂CO₃ pH 9.6 was incubated overnight at 4° C. in Immulon 2HB microtiter plates (Thermo Scientific). Wells were blocked with 150 μL phosphate buffered saline (PBS) with 2% BSA for 1 hour at room temperature. One hundred microliters of biotinylated 14E11 or AB023 (0.7 pmol/L to 6.7 μmol/L) in 90 mmol/L HEPES (4-(2-hydroxyethyl)-1-piperazineethanesulfonic acid) pH 7.2, 100 mmol/L NaCl, 0.1% BSA, 0.1% Tween-20 (HBS [HEPES-buffered saline]) was added, and incubated for 90 minutes at room temperature. After washing with PBS-0.1% Tween-20 (PBS T), 100 μL streptavidin-horse-radish-peroxidase (ThermoFisher Scientific, 1:8000 dilution in FIBS) was added, and incubated at room temperature for 90 minutes. After washing with PBS-T, 100 μL substrate solution (12 mL 30 mmol/L citric acid; 100 mmol/L Na₂HPO₄, pH 5.0; and 1 o-phenylenediamine dihydrochloride tablet, 12 μL 30% H₂O₂) was added. Reactions were stopped after 10 min with 50 μL 2.5 M H₂SO₄. Absorbance at 495 nm was measured on a SpectroMax 340 microplate reader (Molecular Devices, San Jose, Calif.). The data was analyzed by non-linear regression analysis and the apparent K_{d} (equilibrium dissociation constant) was calculated as the concentration of AB023 needed to achieve half-max binding at equilibrium using GraphPad Prism (v. 5.0).

Using this solid phase binding assay, the binding affinity of 14E11 and AB023 for mouse and human FXI were determined. 14E11 bound to mouse FXI with a lower affinity (as previously determined) (Apparent K_{d}˜0.07 nM compared to ˜2-3 pM). 14E11 also demonstrated a lower binding affinity for human FXI and human FXIa than for mouse FM (Apparent K_{d}˜0.39 nM and 0.20 nM respectively) (Table 1). AB023 also demonstrated nanomolar affinity binding for mouse and human FXI (Apparent K_{d} for mFXI ˜0.16 nM, apparent K_{d} for hFXI ˜3.2 nM, and apparent K_{d} for hFXIa ˜1.3 nM), although the binding affinity for both human and mouse FXI was lower than 14E11 (FIG. 4C, Table 1). Taken together, these results confirm that high affinity binding to the same domain was maintained after humanization of the 14E11 antibody.

TABLE 1
Apparent K_{d} Values for 14E11 and AB023

| | Apparent K_{d} (nM) | | |
|---|---|---|---|
| | human FXI | human FXIa | mouse FXI |
| AB023 | 3.2 | 1.3 | 0.16 |
| 14E11 | 0.39 | 0.20 | 0.07 |

A

### 2 Domain Binding Confirmation

In order to confirm that A13023 binds to the apple 2 (A2) domain of FXI, immunoblots using standard techniques were performed (Cheng et al., 2010). FIG. 4A shows immunoblots of recombinant human FXI (lane 1) and human FXI in which the A1, A2, A3, or A4 domain has been replaced with the corresponding domain from prekallikrein (PK), separated by electrophoreses and immunoblotted with AB023, and confirms that AB023, like 14E11, binds the apple 2 (A2) domain of FXI. Fusion proteins were also generated in which each individual apple domain from FXI was fused to tissue plasminogen activator (tPA). The fusion proteins were then separated by electrophoresis and immunoblotted with AB023. FIG. 4B, shows A13023 binds to the A2 domain of FXI.

### Inhibition of FXIIa Activation of FXI

In order to establish that the ability to inhibit FXIIa activation of FXI was maintained by AB023 after humanization of 14E11, an in vitro assay was performed. Briefly, FXI (30 nmol/L) was incubated with 0.5 nmol/L α-FXIIa and dextran sulfate (0.1 μg/mL) at 37° C. in 25 mmol/L HEPES, pH 7.4, 1.50 mmol/L NaCl, and 0.1% BSA in the presence or absence of AB023 (0 nmol/L to 300 nmol/L). After 30 min of incubation, samples were removed and quenched with polybrene (6 μg/mL) to neutralize dextran sulfate and CTI (50 μg/mL) to inactivate FXIIa, after which the generation of FXIa was quantified by measuring rates of S-2366 hydrolysis at 405 nm. Rates of S-2366 hydrolysis were converted to FXIa concentrations using a standard curve. FIG. 4D shows that AB023 concentration-dependently inhibits FXIIa activation of FXI. Like 14E11, AB023 does not inhibit thrombin-mediated activation of FXI (FIG. 4E). In order to determine this an in vitro assay was performed in which FXI (30 nM) was incubated with 2.5 nM α-thrombin and dextran sulfate (0.1 μg/mL) at 37° C. in 25 mM HEPES, pH 7.4, 150 mM NaCl, and 0.1% BSA in the presence or absence of AB023 (300 nM). After 0, 5, 15, 30, or 60 minutes incubation, the samples were removed and quenched with polybrene (6 μg/mL) to neutralize dextran sulfate and hirudin (10 U/mL) to inactivate thrombin, after which the generation of FXIa was quantified by measuring rates of S-2366 hydrolysis at 405 nm. Rates of S-2366 hydrolysis were converted to FXIa concentrations using a standard curve.

Prolong Activated Partial Thromboplastin time (aPTT)

The ability of AB023 to prolong the aPTT in several mammalian plasmas was tested. Pooled plasma from human, baboon, rat, and cynomolgus monkey (90 μL, from three individual subjects)) anticoagulated with 0.38% sodium citrate were mixed with 10 μL of AB023 (0.183-1500 μg/mL) or control (PBS) and allowed to incubate at room temperature for 5 minutes. Forty microliters of the plasma/antibody mixture were then incubated with 40 μL of aPTT reagent (SynthASil, #0020006800, Instrumentation Laboratory, Bedford, Mass.) for 3 minutes at 37° C. After incubation, 40 μL of CaCl₂ was added and time to clot was determined on a KC4™ Analyzer (TCoag, Bray, Ireland). Each sample was assayed in duplicate. For each species tested, AB023 prolonged aPTT in a concentration dependent manner in all species tested (FIG. 4F), the APTT data was expressed as fold change of baseline and plotted again the log₁₀ concentration of AB023.

Taken together, these data demonstrate that the properties of 14E11 were maintained after humanization.

### Anticoagulant Complex Formation

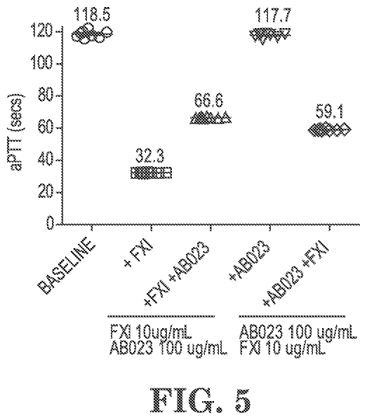

These experiments investigated whether it is the complex that forms between the monoclonal antibody or any binding fragment, variant, or derivative thereof and FXI that is anticoagulant using the activated partial thromboplastin time (aPTT). It is well established in other examples (FIG. 4) that FXI instantly forms a stable immune complex with the high affinity anti-FXI A2 domain antibody, AB023. Here, two separate experiments using FXI deficient plasma from FXI deficient human subjects (George King Bio-medical Inc., Overland Park, Kans.) were performed, the results of which are shown in FIG. 5.

In the first experiment, baseline aPTT was measured in FXI deficient plasma. Forty microliters of plasma was incubated with 40 μL of aPTT reagent (SynthASil, #0020006800, Instrumentation Laboratory, Bedford, Mass.) for 3 minutes at 37° C. After incubation, 40 μL of CaCl₂ was added and time to clot was determined on a KC4™ Analyzer (TCoag, Bray, Ireland). Each sample was assayed eight times. The average baseline aPTT for FXI deficient plasma was 118.5 seconds. Following baseline measurements, FXI (Enzyme Research Laboratories, #HCFXI-1111) was added to 400 μL of FXI deficient plasma for a final concentration of 10 μg/mL, The FXI/FXI-deficient plasma was incubated for 5 min at room temperature and aPTT was measured on 200 μL of the mixture as described above. FIG. 5 shows that addition of purified FXI (MW 160 kD) to FXI deficient plasma reduces the clotting time to 32.3 seconds, as expected, since FXI is the zymogen of the procoagulant enzyme, FXIa. Finally, to the remaining 200 μL of FXI deficient plasma+10 μg/mL AB023 was added for a final concentration of 100 μg/mL (10× excess antibody). This mixture was incubated for 5 min at room temperature and aPTT was measured as described above. Addition of the antibody AB023 (MW 146 kD) in 10 fold molar excess of added FXI to the FXI deficient plasma prolonged the aPTT about 2-fold to an average of 66.6 seconds, consistent with in vitro (FIG. 4) and in vivo (Table 2) experiments in which the AB023 concentration was in excess compared to FXI. The addition of AB023 to normal plasma containing FXI did not prolong the aPTT to the same degree as is observed in FXI deficient plasma. This is likely because the procoagulant activity of FXIa that is generated in the aPTT assay is not inhibited by AB023 (FIG. 4E).

In the second experiment. AB023 was added to 400 μL FXI deficient plasma for a final concentration of 100 μg/mL, incubated for 5 min at room temperature and aPTT was measured on 200 μL of the mixture as described above. Addition of AB023 resulted in no change in the clotting time (mean aPTT=117.7 seconds compared to mean aPTT of FXI deficient plasma of 118.5 seconds), demonstrating that AB023 alone is not anticoagulant without forming a complex with FXI. Lastly, FXI was added to the EXT deficient plasma/AB023 (100 μg/mL) mixture at a final FXI concentration of 10 μg/mL (10-fold excess of antibody to FXI to assure that all FXI goes into an immune complex). The mixture was allowed to incubate at room temperature for 5 minutes and the aPTT was measured as described above. Addition of FXI reduced the aPTT to a mean of 59.1 seconds from 117.7 seconds (FIG. 5).

These experiments indicate that 1) addition of FXI to FXI deficient plasma has a procoagulant effect, 2) AB023 alone does not have an anticoagulant effect, and 3) the immune complex that forms between FXI and AB023 is anticoagulant, but does not produce the equivalent anticoagulant effect of FXI deficiency.

### Characterization of the AB023 Anticoagulant Effect

### Murine Arterial Thrombosis Model

Using a well-established mouse model of experimental arterial thrombosis, the antithrombotic properties of AB023 were determined as had been done previously for 14E11 (Cheng et al., 2010). Briefly, mice were anesthetized with 50 mg/kg IP pentobarbital. The right common carotid artery was exposed and was fitted with a Doppler flow probe. AB023 (1.0 mg/kg, i.v.) was infused into the internal jugular vein 15 min before injury and thrombus formation was induced by applying two 1×1.5 mm filter papers saturated with FeCl₃ (2.5% to 10% solution) to opposite sides of the artery for 3 min. After removal of the pads, the area was irrigated with phosphate buffered saline and flow was monitored for 30 min.

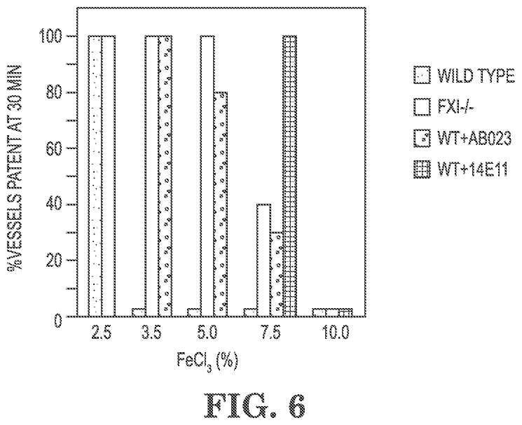

Intravenous infusion of 1.0 mg/kg AB023 into wild-type mice protected mice from carotid occlusion induced by 3.5%, 5.0% and 7,5% FeCl₃ (FIG. 6). These results are comparable to those from FXI−/− mice. The results are consistent with the premise that FXI is activated by Pala in this thrombosis model and demonstrate that die antithrombotic effect was maintained, even in mice, following humanization of the 14E11 antibody.

### PK/PD in Baboon Model

This experiment assessed plasma concentration of AB023 over time and correlated AB023 exposure to aPTT. Six male baboons were dosed via single intravenous bolus injection or single subcutaneous injection with 1.0 mg/kg AB023 on Day 1. Blood was collected at several time points post-administration and was anticoagulated with 0.32% sodium citrate ( 1/10th volume). One aliquot was used to determine plasma AB023 concentration and another aliquot was used for aPTT measurement. To measure aPTT, plasma (40 μL) was incubated with 40 μL of aPTT reagent for 3 minutes at 37° C. After the 3 minute incubation at 37° C., 40 μL of CaCl₂ was added and time to clot was determined on a KC4 Delta™ Analyzer (Tcoag Ireland. Ltd. Wicklow, Ireland). Plasma AB023 concentrations were determined using a partially validated enzyme-linked immunosorbent assay (ELISA) to detect free AB023 in the plasma.

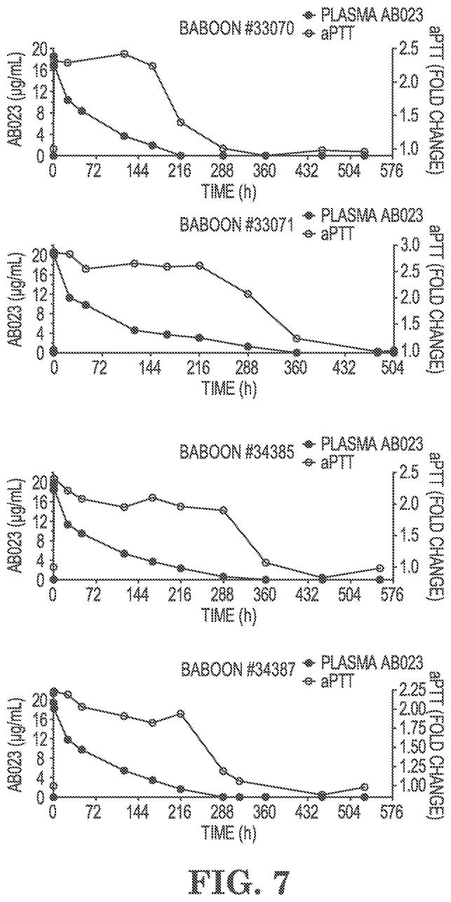

The results showed a rapid and immediate prolongation of aPTT, and an about 2-fold above baseline prolongation of aPTT for at least 1 week (168 h) in all animals. FIG. 7 shows the relationship between AB023 plasma concentrations to aPTT. Similarly, 1.0 mg/kg of AB023 administered SC also resulted in a rapid and immediate prolongation of aPTT. However, after SC administration aPTT remained prolonged approximately 2-fold above baseline for at least 2 weeks (336 h) (not shown). These studies illustrate that aPTT prolongation tracks closely with AB023 exposure.

### Prophylactic Treatment in Baboon Arterial and Venous-Type Thrombosis Models

These studies determined the antithrombotic effect of inhibiting FXI activation by Data using the humanized monoclonal antibody AB023 in a well-established primate model of experimental arterial and venous-type thrombosis.

Non-terminal studies were performed using juvenile male baboons (Papio anubis) weighing 9-13 kg. All studies were approved by the Institutional Animal Care and Use Committee. Each baboon had a healed, surgically placed, chronic exteriorized arterio-venous (AV) shunt connecting the femoral artery and vein, as described elsewhere (Hanson S R, Griffin J H, Harker L A, et al. Antithrombotic effects of thrombin-induced activation of endogenous protein C in primates. J Clin Invest 1993 October; 92(4):2003-12.). Experiments were conducted on non-anticoagulated awake animals that were restrained in a seated position. Anxiety was managed with low dose ketamine not exceeding 2 mg/kg intramuscular; up to once every hour. Experimental treatments were administered through, and blood samples were taken from, a silicone rubber extension tubing incorporated into the AV shunt during the experiments. Red blood cell counts and hematocrit were measured daily in each animal, including before and after experiments, and calculated blood loss did not exceed 4% of the total blood volume on any experimental day. Multiple experiments were performed in the same animals on separate days, with and without treatments. Only animals with shunts that had good unrestricted baseline flow (>250 mL/min) were used in repeat experiments.

In this experimental thrombosis model, acute local thrombosis was initiated with a brief interposed prosthetic modified thrombogenic graft segment within the AV shunt, as previously described (Hanson S R, Griffin J H, Harker L A, et al. Antithrombotic effects of thrombin-induced activation of endogenous protein C in primates. J Clin Invest 1993 October; 92(4):2003-12; Kelly A B, Marzec U M, Krupski W, et al. Hirudin interruption of heparin-resistant arterial thrombus formation in baboons. Blood 1991 Mar. 1; 77(5):1006-12). Since vascular injury exposes flowing blood to the extracellular matrix, which contains structural proteins such as collagen that trigger platelet and FXII activation, we made the graft segments thrombogenic with immobilized collagen coating. The lumens of 20 mm long clinical vascular grafts (expanded-polytetrafluoroethylene, ePTFE, Gore-Tex; W. L. Gore and Associates, Flagstaff, Ariz.) with internal diameters (i.d.) of 4 mm were coated with equine type I collagen (CHRONO-LOG Corporation, Haverton, Pa.) for 15 min, and then dried overnight under sterile airflow. This method produces an even collagen coaling within the graft lumen as determined by scanning electron microscopy (data not shown). The collagen coated (thrombogenic) graft segments were incorporated into silicon rubber tubing, and deployed into the AV shunts in the baboons for the entire duration of 60 min-long acute thrombosis experiments.

The flow of blood through the shunt in non-anticoagulated baboons consistently triggers acute thrombus formation in the collagen-coated ePTFE graft segments. During each experiment, maximum blood flow rate through the grafts (about 250 mL/min) was restricted by distal clamping to 100 mL/min, producing average initial wall shear rates of 265 in 4 mm grafts. Flow rate was continuously monitored using a transgraft ultrasonic flow meter (Transonics Systems, Ithaca, N.Y.). These 4 mm in diameter grafts did not occlude and pulsatile flow rates remained at 100 mL/min during thrombus formation. The graft segment (and thrombus) was removed from the shunt at 60 min or 90 min and the permanent shunt was restored after each experiment. Since thrombus formation was found to extend downstream from the collagen surface over time, platelet accumulation was also measured within a 10 cm-long region of the arteriovenous shunt immediately distal to the graft. This model of thrombus growth on a proximal collagen surface (at the thrombus “head”), with thrombus that propagates distal to the collagen segment (forming a thrombus “tail”).

Thrombus formation was assessed during the 60-90 min-long experiments by quantitative gamma camera imaging of radio-labeled platelets in the graft segment and further assessed by measurement of end-point radio-labeled fibrin deposition after termination of each experiment, as described (1). For quantification of platelet deposition, autologous baboon platelets were labeled with 1 mCi of ¹¹¹In, then re-infused into animals and allowed to circulate for at least 1 hr and up to 4 days before studies were performed. Accumulation of platelet-associated radioactivity onto grafts was determined at 5 min intervals using a GE-400A-61 gamma scintillation camera interfaced with a NuQuest InteCam computer system as described for other devices in the chronic AV shunt model (Gruber and Hanson, Blood 2003; 102:953-955; Gruber et al., Thromb Res 2007; 119:121-127; Hanson et al., J Clin Invest 1993; 92:2003-2012; Tucker et al., Blood 2009; 113:936-944). At 60-90 minutes, the graft was removed, rinsed, dried, and stored refrigerated for subsequent evaluation of ¹²⁵I-fibrin content, as previously described (Gruber and Hanson, Blood 2003; 102:953-955; Hanson et al., J Clin Invest 1993; 92:2003-2012). Briefly, homologous ¹²⁵I-labeled baboon fibrinogen (5-25 μg, 4 μCi, >90% clottable) was injected i.v. 10 min before each study. Incorporation of labeled fibrinogen/fibrin into the thrombus was assessed using a gamma counter (Wizard-3, PerkinElmer, Shelton. C T) at least 30 days after removal of the graft from the AV shunt to allow ¹¹¹In attached to platelets to decay. ¹²⁵I-radioactivity deposited in the graft was measured and compared to the radioactivity of dutiable fibrin (ogen) in plasma samples taken during the original study.

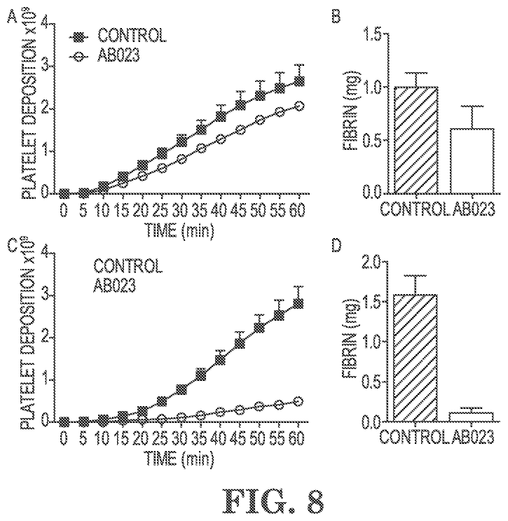

In the first experiment (FIG. 8), a collagen coated 4 mm in diameter vascular graft segment with a 9 mm in diameter silicon chamber to mimic venous flow was positioned 20 mm downstream was used to test AB023 antithrombotic effect (0.2 ma/kg, i.v. administered 1 hour prior to the experiment). A total of 4 non-terminal studies were performed using 2 juvenile male baboons and the data were compared to the controls used in an identical 14E11 experiment (n=7 experiments/group in 4 animals for control group).

The results from this study are shown in FIGS. 8A-I). The panels to the left of the figures show platelet deposition and the panels to the right show terminal fibrin deposition. This study showed platelet accumulation rates within the vascular grafts appeared to be slightly lower in AB023 treated animals than in untreated controls (FIG. 8A), while near complete inhibition of platelet accumulation was achieved within the expansion chamber in AB023-treated baboons (FIG. 8C). These data are similar to what was observed after 14E11 administration, and demonstrate that the antithrombotic effect of AB023 is comparable to the murine precursor 14E11. Fibrin deposition was also lower in both arterial-type and venous-type thrombosis (FIGS. 8B and D).

In a second set of experiments, acute local thrombosis was initiated with a 20 mm-long, 4 mm internal diameter collagen coated prosthetic vascular graft segments deployed into the chronic AV shunts for 60 minutes in 8 non-terminal experiments using 4 juvenile male baboons. Baboons were treated with either AB023 (1.0 mg/kg, i.v. injection or) or saline (control, i.v.). Antithrombotic effects of AB023 assessed 24 h after i.v. treatment.

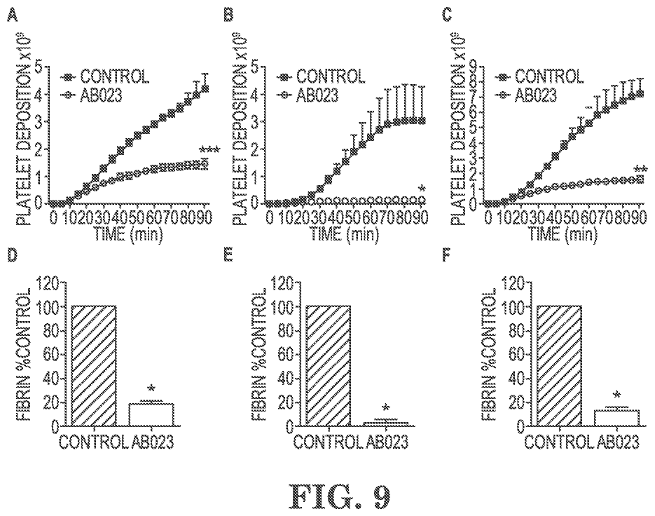

Platelet accumulation within the collagen coated vascular grafts was lower in A13023 treated animals (FIG. 9A), and near complete inhibition of platelet accumulation was achieved in the region 10 cm immediately downstream of the graft (the “tail”) by AB023 (FIG. 9B). Overall, total platelet accumulation (platelet accumulation within the thrombogenic graft plus the tail) was reduced after pretreatment with A13023 (1.0 mg/kg, i.v., FIG. 9C). These studies showed that AB023, like its murine precursor, is antithrombotic in the primate model of thrombosis. Furthermore, at the higher dose of 1.0 mg/kg, AB023 was effective in attenuating arterial-type thrombus growth rates in the thrombogenic graft as well as in “the tail”. Fibrin deposition in both the graft and “tail” was decreased after AB023 treatment.

APTT was monitored throughout the study and was prolonged after AB023 treatment after infusion and remained elevated beyond 24 hours post-treatment in the A13023 treated group (30 min and 60 min into the experiment, Table 2), while no change in aPTT was observed in the control group. PT was also measured throughout the course of the study. AB023 did not alter the PT compared to controls (Table 2).

TABLE 2
aPTT and PT from baboon vascular thrombosis model

| | aPTT (s) | | |
|---|---|---|---|
| | Pre-study | 30 min | 60 min |
| control | 33.2 ± 3.2 | 33.4 ± 3.2 | 32.7 ± 1.8 |
| AB023 | 78.4 ± 11.0 | 78.5 ± 12.4 | 78.7 ± 12.9 |

| | PT (s) | | |
|---|---|---|---|
| | Pre-study | 60 min | 24 h |
| control | 8.9 ± 0.1 | 8.8 ± 0.2 | — |
| AB023 | 9.2 ± 0.1 | 9.0 ± 0.4 | 9.1 ± 0.4 |

Data presented are mean ± standard deviation.

These data demonstrate that the humanized AB023 antibody maintained its anticoagulant properties, and reduced experimental thrombosis in both mouse and non-human primate models of thrombosis.

### Comparison of 14E11 and AB023

Following humanization, the anticoagulant effect of A13023 was compared to 14E11 using the aPTT assay as described above, showing A8023 similarly prolonged the aPTT in human and baboon plasma compared to 14E11 (FIG. 3).

The effect of 14E11 or AB023 on contact pathway activation, FXI autoactivation, and reciprocal FXII activation by FXIa was also examined. Briefly, in order to compare the effects of 14E11 and AB023 on contact pathway activation, FXI (80 nM) in HEPES (20 mM HEPES, pH 7.4, 100 mM NaCl, 0.1% PEG-8000 was incubated at 37° C. for 15 min with or without 1280 nM 14E11 or 800 nM A13023. At time zero, FXIIa, HK, and dextran sulfate, all in HEPES were added so that final concentrations were FXI (30 nM), FXIIa (5 nM), HK (30 dextran sulfate (0.1 μg/ml), and 480 nM 14E11 or 300 nM AB023. At various time points, 5 μL aliquots were supplemented with corn trypsin inhibitor (CTI) (500 nM final concentration) and Polybrene 20 μg/ml (final), and ΔOD_{405 nm} was followed on a microplate reader in the presence of 250 μM S-2366.

In order to compare the effects of 14E11 and AB023 on FXIa autoactivation, purified human FXI was mixed with DXS (0.1 μM) in the presence of 25 and 100 nM 14E11 or AB023 or purified human FXI was mixed with purified leukocyte-derived DNA in the presence of various concentrations of 14E11 (0-100 nM) or AB023 (0-100 nM) in 20 mM HEPES, pH 7.4, 100 mM NaCl, 0.1% PEG-8000, ZnCl₂ (10 μM) at 37° C. for 60 min. FXI activation was terminated by mixing aliquots with polybrene (0.2 mg/mL) and FXIa amidolytic activity was measured using the chromogenic substrate S-2366 (1 mM) and Vₘₐₓ was measured as OD 405 nm/min.

Finally, in order to compare the effects of 14E11 and AB023 on reciprocal FXII activation by FXIa, purified human FXII (200 nM) and FXIa (10 nM) were incubated with 14E11 or AB023 (0-200 nM) in 20 mM HEPES, pH 7.4, 100 mM NaCl, 0.1% PEG-8000, ZnCl₂ (10 μM) at 37° C. for 60 min. FXII activation was stopped by aprotinin (1.0 μM) and FXIIa amydolitic activity was measured using the chromogenic substrate S-2302 (1 mM), and Vₘₐₓ was measured as OD 405 nm/min.

Despite an apparent lower affinity for FXI, AB023 was more effective at inhibiting FXIIa activation of FXI (FIG. 10C). In addition, attenuation of FXI autoactivation in the presence of a negative surface (dextran sulfate or DNA) by AB023 was greater compared to 14E11 (FIG. 10A-B).

Humanization of 14E11 into AB023 resulted in a novel binding molecule that has, for example, unexpected gain of function, i.e., 2-way inhibitory activity on the interactions of FXII and FXI (FIGS. 10C, D). FIG. 10C shows FXI activation by FXII a. FIG. 10D shows the respective inhibitory effects of the anti-FXI IgGs on FXII activation by FXIa when human FXII (200 nM) was activated by 10 nM human FXIa in the presence of 14E11 or AB023. Not only is AB023 more effective at inhibiting FXIIa activation of FXI than its murine precursor (FIG. 10C) but AB023 inhibits reciprocal activation process between FXII and FXI while 14E11 does not. The unexpected inhibitory gain may be related to sequence homology between the molecules. Blast alignment showed sequence homology between 14E11 and AB023 to be about 60-70% (data not shown).

### LISTED SEQUENCES

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

LC CDR 1
SEQ ID NO: 1
KASQDVSTAVA
Lc CDR 2
SEQ ID NO: 2
LTSYPNT
LC CDR 3
SEQ ID NO: 3
QQHYKTPYS
HC CDR 1
SEQ ID NO: 4
GYGIY
HC CDR 2
SEQ ID NO: 5
MIWGDGRTDYNSALKS
HC CDR 3
SEQ ID NO: 6
DYYGSKDY
FX1 with A2 domain in bold
SEQ ID NO: 7
MIFLYQVVHFILFTSVSGECVTQLLKDTCFEGGDITTVFTPSAKYCQVVCTYHPRCLLFTFTAESPSEDPT
RWFTCVLKDSVTETLPRVNRTAAISGYSFKQCSHQISSACNKDIYVDLDMKGINYNSSVAKSAQECQERCTD
DVHCHFFTYATRQFSLEHRNICLLKHTQTGTPTRITKLDKVVGFSLKSCALSNLACIRDIFPNTFFADS
NIDSVMAPDAFVCGRICTHHPGCLFFTFFSQEWPKESQRNLCLLKTESGLPSTRIKKSKALSGFSLQSCR
HSIPVFCHSSFYHDTDFLGEELDIVAAKSHEACQKLCTNAVRCQFFTYTPAQASCNEGKGKCYLKLSSNGS
PTKILHGRGGISGYTLRLCKMDNECTTKIKPRIVGGTASVRGEWWQVTLHTTSPTQRHLCGGSIIGNQWI
LTAAHCFYGVESPKILRVYSGILNQSEIKEDTSFFGVQEIIIHDQYKMAESGYDIALLKLETTVNYTDSQR
PICLPSKGDRNVIYTDCWVTGWGYRKLRDKIQNTLQKAKIPLVTNEECQKRYRGHKITHKMICAGYREGGK
DACKGDSGGPLSCKHNEVWHLVGITSWGEGCAQRERPGVYTNVVEYVDWILEKTQAV
VH domain of AB023
SEQ ID NO: 8
QVQLQESGPGLVKPSETLSITCTVSGFSLTGYGIYWVRQPPGKGLEWLGMIWGDGRTDYNSALKSRVTISK
DNSKSQVSLKLSSVTAADTARYYCARDYYGSKDYWGQGTTVTSS
VL domain of AB023
SEQ ID NO: 9
DIQMTQSPSSLSASVGDRVTITCKASQDVSTAVAWYQQKPGKAPKLLIYLTSYRNTGVPDRFSGSGSGTDF
TFTISSLQPEDIAVYYCQQHYKTPYSFGGGTKVEIK
LC of AB023
SEQ ID NO: 10
DIQMTQSPSSLSASVGDRVTITCKASQDVSTAVAWYQQKPGKAPKLLIYLTSYRNTGVPDRFSGSGSGTDF
TFTISSLQPEDIAVYYCQQHYKTPYSFGGGTKVEIKRTVAAPSVFIFPPSDEQLKSGTASVVCLLNNFYPR
EAKVQWKVDNALQSGNSQESVTEQDSKDSTYSLSSTLTLSKADYEKHKVYACEVTHQGLSSPVTKSFNRGE
C
HC of AB023
SEQ ID NO: 11
QVQLQESGPGLVKPSETLSITCTVSGFSLTGYGIYWVRQPPGKGLEWLGMIWGDGRTDYNSALKSRVTISK
DNSKSQVSLKLSSVTAADTARYYCARDYYGSKDyWGQGTTVTVSSASTKGPSVFPLAPCSRSTSESTAALG
CLVKDYFPEPVTVSWNSGALTSGVHTFPAVLQSSGLYSLSSVVTVPSSSLGTKTYTCNVDHKPSNTKVDKR
VESKYGPPCPPCPAPEFLGGPSVFLFPPKPKDTLMISRTPEVTCVVVDVSQEDPEVQFNWYVDGVEVHNAK
TKPREEQFNSTYRVVSVLTVLHQDWLNGKEYKCKVSNKGLPSSIEKTISKAKGQPREPQVYTLPPSQEENT
KNQVSLTCLVKGFYPSDIAVEWESNGQPENNYKTTPPVLDSDGSFFLYSRLTVDKSRWQEGNVFSCSVMHE
ALHNHYTQKSLSLSLGK
LC domain of AB023 DNA encoding SEQ ID NO: 10)
SEQ ID NO: 12
ATGAGGGTCCCCGCTCAGCTCCTGGGGCTCCTGCTGCTCTGGCTCCCAGGCGCGCGATGTGACATCCAGAT
GACCCAGTCTCCATCCTCCCTGTCTGCATCTGTAGGAGACAGAGTCACCATCACTTGCAAGGCCAGTCAGG
ATGTGAGTACTGCTGTTGCCTGGTATCAGCAGAAACCAGGGAAAGCCCCTAAGCTCCTGATCTACTTGACA
TCCTACCGGAACACTGGGGTCCCAGATAGGTTCAGTGGAAGTGGATCTGGGACAGATTTTACTTTCACCAT
CAGCAGCCTGCAGCCTGAAGATATTGCAGTTTATTACTGTCAGCAACATTATAAAACTCCGTATTCGTTCG
GCGGAGGGACCAAGGTGGAAATCAAACGAACTGTGGCTGCACCATCTGTCTTCATCTTCCCGCCATCTGAT
GAGCAGTTGAAATCTGGAACTGCCTCTGTTGTGTGCCTGCTGAATAACTTCTATCCCAGAGAGGCCAAAGT
ACAGTGGAAGGTGGATAACGCCCTCCAATCGGGTAACTCCCAGGAGAGTGTCACAGAGCACGACAGCAAGG
ACAGCACCTACAGCCTCAGCAGCACCCTGACGCTGAGCAAAGCAGACTACGAGAAACACAAAGTCTAGGCC
TGCGAAGTCACCCATCAGGGCCTGAGCTCGCCCGTCACAAAGAGCTTCAACAGGGGAGAGTGTTAG
HC domain of AB023 DNA encoding SEQ ID NO: 11)
SEQ ID NO: 13
ATGGGTTGGAGCCTCATCTTGCTCTTCCTTGTCGCTGTTGCTACGCGTGTCCACTCCCAGGTGCAGCTGCA
GGAGTCGGGCCCAGGACTGGTGAAGCCTTCGGAGACCCTGTCCATCACCTGCACTGTCTCTGGGTTCTCAT
TAACCGGCTATGGTATATACTGGGTGCGGCAGCCCCCAGGGAAGGGACTGGAGTGGCTGGGCTATGATATGG
GGTGATGGAAGAACAGACTATAATTCAGCTCTCAAATCCCGAGTCACCATATCAAAGGACAACTCCAAGAG
CCAGGTGTCCCTGAAGCTGAGCTCTGTGACCGcrGCGGACACGGCCAGGTATTACTGTGCGAGAGATTACT
ACGGTAGTAAGGACTACTGGGGCCAGGGCACCACGCTCACCGTCTCCTCAGCTTCCACCAAGGGCCCATCC
GTCTTCCCCCTGGCGCCCTGCTCCAGGAGCACCTCCGAGAGCACAGCCGCCCTGGGCTGCCTGGTCAAGGA
CTACTTCCCCCAACCCGTCACCGTCTCGTGGAACTCAGGCGCCCTCACCAGCGCCGTCCACACCTTCCCGG
CTGTCCTACAGTCCTCAGGACTCTACTCCCTCAGCAGCGTGGTGACCGTGCCCTCCAGCAGCTGGGCACG
AAGACCTACACCTGCAATGTAGATCACAAGCCCAGCAACACCAAGGTGGACAAGAGAGTTGAGTCCAAATA
TGGTCCCCCATGCCCACCATGCCCAGCACCTGAGTTCCTGGGGGGACCATCAGTCTTCCCTGTTCCCCCCAA
AACCCAAGGACACTCTCATGATCTCCCGGACCCCTGAGGTCACGTGCGTGGTGGTGGACGTGAGCCAGGAA
GACCCCGAGGTCCAGTTCAACTGGTACGTGGATGGCGTGGAGGTGCATAATGCCAAGACAAAGCCGCGGGA
GGAGCAGTTCAACAGCACGTACCGTGTGCTCACCGTCCTCACCGTCCTGCACCAGGACTGGCTGAACGGCA
AGGAGTACAAGTSCAAGGTCTCCAACTAAAGGCCTCCCGTCCTCCATCGAGWACCATCTCCAAAGCCAAA
GGGCAGCCCCGAGAGCCACAGGTGTACACCCTGCCCCCATCCCAGGAGGAGATGACCAAGAACCAGGTCAG
CCTGACCTGCCTGGTCAAAGGCTTCTACCCCAGCGACATCGCCGTGGAGTGGGAGAGCAATGGGCAGCCGG
AGAACAACTACAAGACCACGCCTCCCGTGCTGGACTCCGACGGCTCCTTCTTCCTCTACAGCAGGCTAACC
GTGGACAAGAGCAGGTGGCAGGAGGGGAATGTPCTTCTCATGCTCCGTGATGCATGAGGCTCTGCACAACCA
CTACACACAGAAGAGCCTCTCCCTGTCTCTGGGTAAATGA

The foregoing discussion of the disclosure has been presented for purposes of illustration and description. The foregoing is not intended to limit the disclosure to the forms or forms disclosed herein. For example, various features of the disclosure are grouped together in one or more aspects, embodiments, and configurations for the purpose of streamlining the disclosure and ease of understanding. The features of the aspects, embodiments, and configurations of the disclosure may be combined in alternate aspects, embodiments, and configurations other than those discussed. This disclosure, thus, is not to be interpreted as reflecting an intention that the claimed disclosure requires more features than are expressly recited in each claim. Rather, as the following claims reflect, invention aspects lie in less than all features of a single foregoing disclosed aspect, embodiment, and configuration. Thus, the following claims are hereby incorporated into this Detailed Description, with each claim standing on its own as a separate preferred embodiment of the disclosure. Moreover, though the description of the disclosure has included description of one or more aspects, embodiments, or configurations and certain variations and modifications, other variations, combinations, and modifications are within the scope of the disclosure, for example, as may be within the skill and knowledge of those in the art, after understanding the present disclosure. It is intended to obtain rights which include alternative aspects, embodiments, and configuration to the extent permitted, including alternate, interchangeable and/or equivalent structures, functions, ranges, or steps to those claimed, whether or not such alternate interchangeable and/or equivalent structures, functions, ranges or steps are disclosed herein, and without intending to publicly dedicate any patentable subject matter.

What is claimed is:

1. A monoclonal antibody, or an antigen-binding fragment, variant, or derivative thereof, which specifically binds human Factor XI and/or human Factor XIa comprising:
a CDR1 of the light chain comprising the sequence KASQDVSTAVA (SEQ ID NO: 1);
a CDR2 of the light chain comprising the sequence LTSYRNT (SEQ ID NO: 2);
a CDR3 of the light chain comprising the sequence QQHYKTPYS (SEQ ID NO: 3);
a CDR) of the heavy chain comprising the sequence GYGIY (SEQ ID NO: 4);
a CDR2 of the heavy chain comprising the sequence MIWGDGRTDYNSALKS (SEQ ID NO: 5);
a CDR3 of the heavy chain comprising the sequence DYYGSKDY (SEQ ID NO: 6).

2. A monoclonal antibody, or an antigen-binding fragment, variant, or derivative thereof; according to claim 1, comprising a V_{E} region as set forth in SEQ ID NO: 8

3. A monoclonal antibody, or an antigen-binding fragment, variant, or derivative thereof, according to claim 1, wherein the monoclonal antibody comprises a V_{L} region as set forth in SEQ NO: 9.

4. A monoclonal antibody, or an antigen-binding fragment, variant, or derivative thereof, according to claim 1, wherein the monoclonal antibody comprises a region as set forth in SEQ ID NO: 8 and a V_{L} region as depicted in SEQ ID NO: 9.

5. A monoclonal antibody, or an antigen-binding fragment, variant, or derivative thereof, according to claim 1, wherein the monoclonal antibody or binding fragment thereof comprises a light chain as depicted in SEQ ID NO: 10 or encoded by SEQ ID NO: 12.

6. A monoclonal antibody, or an antigen-binding fragment, variant, or derivative thereof, according to claim 1, wherein the monoclonal antibody or binding fragment thereof comprises a heavy chain as depicted in SEQ ID NO: 11 or encoded by SEQ ID NO: 13.

7. A monoclonal antibody, or an antigen-binding fragment, variant, or derivative thereof, according to claim 1, wherein the monoclonal antibody, or binding fragment, variant, or derivative thereof comprises a light chain as depicted in SEQ ID NO: 10 or encoded by SEQ ID NO: 12 and a heavy chain as depicted in SEQ ID NO: 11 or encoded by SEQ ID NO: 13.

8. A monoclonal antibody, or an antigen-binding fragment, variant, or derivative thereof, according to claim 1, wherein the monoclonal antibody is a humanized monoclonal antibody or a chimeric monoclonal antibody.

9. A monoclonal antibody, or an antigen-binding fragment, variant, or derivative thereof, according to claim 1, wherein the antibody is an IgG.

10. A monoclonal antibody, or an antigen-binding fragment, variant, or derivative thereof, according to claim 1, wherein the antibody is an IgG4.

11. A polynucleotide encoding the monoclonal antibody, an antigen-binding fragment, a variant, or a derivative as defined in claim 1.

12. A vector comprising the polynucleotide of claim 11.

13. The vector according to claim 12, wherein the vector is an expression vector.

14. A culture comprising a host cell that comprises the nucleic acid according to claim 11.

15. A process for the production of a monoclonal antibody, or an antigen-binding fragment, variant, or derivative thereof, the process comprising culturing the host cell defined in claim 14 under conditions allowing the expression of the monoclonal antibody, the antigen-binding fragment, the variant, or the derivative thereof.

16. A pharmaceutical composition comprising the monoclonal antibody, or the antigen-binding fragment, variant, or derivative thereof, produced according to the process of claim 15, and a pharmaceutically acceptable excipient.

17. A pharmaceutical composition comprising a therapeutically effective amount of the monoclonal antibody, or the antigen-binding fragment, variant, or derivative thereof, according to claim 1 and a pharmaceutically acceptable excipient.

18. The pharmaceutical composition according to claim 17, further comprising one or more additional active agents.

19. The pharmaceutical composition according to claim 18, wherein the further additional active agent is selected from the group comprising plasminogen activators (thrombolytics/fibrinolytics); inhibitors of plasminogen activators; inhibitors of thrombin-activated fibrinolysis inhibitors (TAFI) including tissue plasminogen activator (t-PA), streptokinase, reteplase, and urokinase; non-fractionated heparins; low molecular weight heparins; heparinoid; hirudin: bivalirudin; and argatroban.

20. A method of using the monoclonal antibody, or an antigen-binding fragment, variant, or derivative thereof; according to claim 1 as a medicament comprising administering a therapeutically effective amount of the monoclonal antibody, antigen-binding fragment, variant, or derivative thereof to a subject in need of the medicament.

21. The method of claim 20, wherein the subject needs treatment and/or prophylaxis of thrombotic or thromboembolic disorders, thrombotic or thromboembolic complications, or inflammatory disorders.

22. The method of claim 20, wherein the subject needs treatment and/or prophylaxis for inhibiting blood coagulation, platelet aggregation, and/or thrombosis.

23. A method of using the monoclonal antibody, or an antigen-binding fragment, variant, or derivative thereof, according to claim 1 as an anticoagulant in blood samples, blood preservations, plasma products, biological samples, or medicinal additives or devices, comprising adding the monoclonal antibody or antigen-binding fragment thereof to a sample in need of the anticoagulant.

24. A kit comprising the monoclonal antibody, or an antigen-binding fragment, variant, or derivative thereof, according to claim 1, and directions for the use thereof.

25. The kit according to claim 21, wherein the monoclonal antibody, or an antigen-binding fragment, variant, or derivative thereof; is in lyophilized form, and further comprising a pharmaceutically acceptable carrier for resuspension of the monoclonal antibody, antigen-binding fragment, variant, or derivative thereof.

26. The process of claim 15 further comprising recovering the produced antibody, antigen-binding fragment, variant, or derivative thereof, from the culture.
